(19) United States
(12) Patent Application Publication
(10) Pub. No.: US 2013/0137753 A1
(43) Pub. Date: May 30, 2013

(54) METHODS AND COMPOSITIONS FOR THE DIAGNOSIS AND TREATMENT OF BREAST CANCER

(71) Applicants: Lalita Samant, Mobile, AL (US); Rajeev Samant, Mobile, AL (US)

(72) Inventors: Lalita Samant, Mobile, AL (US); Rajeev Samant, Mobile, AL (US)

(73) Assignee: UNIVERSITY OF SOUTH ALABAMA, Mobile, AL (US)

(21) Appl. No.: 13/813,921

(22) PCT Filed: Aug. 2, 2011
(86) PCT No.: PCT/US11/46336

§ 371 Date: Feb. 1, 2013

Related U.S. Application Data
(60) Provisional application No. 61/400,823, filed on Aug. 3, 2010.

(51) Int. Cl. C12Q 1/68; G01N 33/68

(52) U.S. Cl. 514/44 A; 435/7.92; 506/9; 530/389.7; 514/44 R; 435/6.12

(57) ABSTRACT

Embodiments of the present disclosure relate to methods and compositions for the diagnosis and treatment of breast cancer. In some embodiments, the present disclosure relates to the use of Merlin, OPN and particular microRNAs for evaluating the presence of breast cancer in a subject and for identifying therapeutic compounds.

### RELATED APPLICATIONS

This is a non-provisional application claiming priority to U.S. Provisional Application No. 61/400,823 filed Aug. 3, 2010, the contents of which is incorporated herein by reference in its entirety.

### STATEMENT REGARDING FEDERALLY SPONSORED R&D

This invention was made with government support under NIH Grant/Contract Numbers 1RO1CA138850 and 1RO1CA140472. The government has certain rights in the invention.

### REFERENCE TO SEQUENCE LISTING

The present application is being filed along with a Sequence Listing in electronic format. The Sequence Listing is provided as a file entitled USA_{—}009WO.TXT, created Aug. 1, 2011, which is approximately 32 KB in size. The information in the electronic format of the Sequence Listing is incorporated herein by reference in its entirety.

### FIELD OF THE INVENTION

Embodiments of the present disclosure relate to methods and compositions for the diagnosis and treatment of breast cancer. In some embodiments, the present disclosure relates to the use of Merlin, OPN and particular microRNAs for evaluating the presence, absence or metastatic potential of breast cancer in a subject and for identifying therapeutic compounds.

### BACKGROUND

Breast cancer is the most common cancer and the second cause of cancer death in women. Worldwide, breast cancer comprises 22.9% of all cancers in women. In 2008, breast cancer caused 458,503 deaths worldwide (13.7% of cancer deaths in women). Breast cancer is more than 100 times more common in women than breast cancer in men, although men tend to have poorer outcomes due to delays in diagnosis.

Risk factors for breast cancer include race, age, and mutations in the tumor suppressor genes BRCA-1 and -2 and p53. Alcohol consumption, fat-rich diet, lack of exercise, exogenous post-menopausal hormones and ionizing radiation also increase the risk of developing breast cancer. Estrogen receptor (ER) and progesterone receptor (PR) negative breast cancer, large tumor size, high grade cytology and age below 35 years are associated with a negative prognosis (Goldhirsch et al., (2001) J. Clin. Oncol. 19: 3817-27, incorporated by reference in its entirety).

Current therapeutic options for treatment of breast cancer, including metastatic breast cancer, include surgery (e.g. resection, autologous bone marrow transplantation), radiation therapy, chemotherapy (e.g. anthracyclines such as doxorubicin, alkylating agents such as cyclophosphamide and mitomycin C, taxanes such as paclitaxel and docetaxel, antimetabolites such as capecitabine, microtubule inhibitors such as the vinca alkaloid navelbine), endocrine therapy (e.g. antiestrogens such as tamoxifen, progestins such as medroxyprogesterone acetate and megastrol acetate, aromatase inhibitors such as aminoglutethamide and letrozole) and biologics (e.g. cytokines, immunotherapeutics such as monoclonal antibodies). Most commonly metastatic breast cancer is treated by one or a combination of chemotherapy (the most effective drugs including cyclophosphamide, doxorubicin, navelbine, capecitabine and mitomycin C) and endocrine therapy.

In spite of considerable research into therapies, breast cancer remains difficult to diagnose and treat effectively. Accordingly, there is a need in the art for improved methods for detecting and treating such cancers.

### SUMMARY

Embodiments of the present disclosure relate to methods and compositions for the diagnosis and treatment of breast cancer. In some embodiments, the present disclosure relates to the use of Merlin, OPN and particular microRNAs for evaluating the presence, absence or metastatic potential of breast cancer in a subject and for identifying therapeutic compounds. Some embodiments include methods for evaluating the presence, absence or metastatic potential of a breast cancer in a subject comprising measuring the expression level of Merlin protein in a sample obtained from the subject.

Some embodiments also include comparing the expression level of Merlin in the sample to the expression level of Merlin protein in normal tissue, or cancerous tissue with a known metastatic potential.

In some embodiments, a decrease in the level of expression of Merlin is indicative of the presence or metastatic potential of the breast cancer.

Some embodiments also include measuring the expression level of a nucleic acid encoding OPN or the expression level of OPN protein in the sample.

In some embodiments, the expression level of a nucleic acid encoding OPN is measured in the sample.

In some embodiments, the expression level of OPN protein is measured in the sample.

In some embodiments, an increase in the expression level of a nucleic acid encoding OPN or expression level of OPN protein relative to a pre-determined expression level of a nucleic acid encoding OPN or expression level of OPN protein is indicative of the presence or metastatic potential of the breast cancer.

In some embodiments, the breast cancer comprises an infiltrating ductal carcinoma (IDC).

In some embodiments, the breast cancer comprises a distant metastasis.

In some embodiments, the sample comprises a protein sample removed from the subject's body, and wherein the expression level of Merlin protein is measured outside the subject's body.

In some embodiments, the subject is mammalian.

In some embodiments, the subject is human.

Some embodiments include methods for evaluating the presence, absence or metastatic potential of a breast cancer in a subject comprising measuring the expression level of a phosphorylated Merlin protein in a sample obtained from the subject.

Some embodiments also include comparing the expression level of phosphorylated Merlin in the sample to the expression level of phosphorylated Merlin protein in normal tissue, or cancerous tissue with a known metastatic potential.

In some embodiments, an increase in the level of expression of phosphorylated Merlin is indicative of the presence or metastatic potential of the breast cancer.

Some embodiments also include measuring the expression level of a nucleic acid encoding OPN or the expression level of OPN protein in the sample.

In some embodiments, the expression level of a nucleic acid encoding OPN is measured in the sample.

In some embodiments, the expression level of OPN protein is measured in the sample.

In some embodiments, an increase in the expression level of a nucleic acid encoding OPN or expression level of OPN protein relative to a pre-determined expression level of a nucleic acid encoding OPN or expression level of OPN protein is indicative of the presence or metastatic potential of the breast cancer.

In some embodiments, the breast cancer comprises an infiltrating ductal carcinoma (IDC).

In some embodiments, the breast cancer comprises a distant metastasis.

In some embodiments, the sample comprises a protein sample removed from the subject's body, and wherein the expression level of phosphorylated Merlin protein is measured outside the subject's body.

In some embodiments, the phosphorylated Merlin protein is phosphorylated at Threonine 230, Serine 315, or at both residues.

In some embodiments, the subject is mammalian.

In some embodiments, the subject is human.

Some embodiments include methods for evaluating the presence, absence or metastatic potential of a breast cancer in a subject comprising measuring the expression level of a nucleic acid encoding OPN or the expression level of OPN protein in a sample obtained from the subject.

Some embodiments also include comparing the expression level of a nucleic acid encoding OPN or the expression level of OPN protein in the sample to the expression level of a nucleic acid encoding OPN or the expression level of OPN protein in normal tissue, or cancerous tissue with a known metastatic potential.

In some embodiments, an increase in the level of expression of a nucleic acid encoding OPN or the level of expression of OPN protein is indicative of the presence or metastatic potential of the breast cancer.

In some embodiments, the breast cancer comprises an infiltrating ductal carcinoma (IDC).

In some embodiments, the breast cancer comprises a distant metastasis.

In some embodiments, the sample comprises a protein sample removed from the subject's body, and wherein the expression level of a nucleic acid encoding OPN or the expression level of OPN protein is measured outside the subject's body.

In some embodiments, the subject is mammalian.

In some embodiments, the subject is human.

Some embodiments include methods for identifying a therapeutic compound comprising: contacting a target cell with a test compound, wherein the cell comprises a breast cancer cell; and determining whether the test compound significantly changes the level of Merlin protein.

Some embodiments also include comparing the level of Merlin protein in a target cell which has not been contacted with the test compound to the level of Merlin protein in a target cell contacted with the test compound.

Some embodiments also include determining whether the test compound increases the level of Merlin protein.

Some embodiments also include determining whether the test compound decreases the expression level of a nucleic acid encoding OPN or OPN protein.

In some embodiments, the target cell comprises an infiltrating ductal carcinoma (IDC) cell.

In some embodiments, the target cell comprises a distant metastasis cell.

In some embodiments, the target cell is mammalian.

In some embodiments, the target cell is human.

Some embodiments include methods for identifying a therapeutic compound comprising: contacting a target cell with a test compound, wherein the cell comprises a breast cancer cell; and determining whether the test compound significantly changes the expression level of a nucleic acid encoding OPN or the level of expression of OPN protein.

Some embodiments also include comparing the expression level of a nucleic acid encoding OPN or the level of expression of OPN protein in a target cell which has not been contacted with the test compound to the expression level of a nucleic acid encoding OPN or the level of expression of OPN protein in a target cell contacted with the test compound.

Some embodiments also include determining whether the test compound decreases the expression level of a nucleic acid encoding OPN or the level of expression of OPN protein.

Some embodiments also include determining whether the test compound increases the expression level of Merlin protein.

In some embodiments, the target cell comprises an infiltrating ductal carcinoma (IDC) cell.

In some embodiments, the target cell comprises a distant metastasis cell.

In some embodiments, the target cell is mammalian.

In some embodiments, the target cell is human.

Some embodiments include kits for evaluating the presence, absence or metastatic potential of a breast cancer in a subject comprising a detection reagent that binds to Merlin protein.

Some embodiments also include a detection reagent that binds to a nucleic acid encoding OPN or that binds to OPN protein.

In some embodiments, the breast cancer comprises an infiltrating ductal carcinoma (IDC).

In some embodiments, the breast cancer comprises a distant metastasis cell.

In some embodiments, the subject is mammalian.

In some embodiments, the subject is human.

Some embodiments include methods for evaluating the presence, absence, or metastatic potential of a cancer in a subject comprising: measuring the expression level of at least one microRNA in a sample obtained from the subject, wherein the microRNA comprises at least about 80% identity to a sequence selected from the group consisting of SEQ ID NO.s:01-74, and a fragment comprising at least 10 consecutive nucleotides thereof.

Some embodiments also include comparing the expression level of the microRNA in the sample to the expression level of the microRNA in normal tissue, or cancerous tissue with a known metastatic potential.

In some embodiments, an increase in the level of expression of said microRNA is indicative of an increased metastatic potential.

In some embodiments, the microRNA is selected from the group consisting of SEQ ID NO.s:01-60, and SEQ ID NO:61.

In some embodiments, a decrease in the level of expression of said microRNA is indicative of an increased metastatic potential.

In some embodiments, the microRNA is selected from the group consisting of SEQ ID NO.s:62-73, and SEQ ID NO:74.

In some embodiments, the microRNA targets Merlin.

In some embodiments, the microRNA is selected from the group consisting of SEQ ID NO:01, SEQ ID NO:17, SEQ ID NO:19, SEQ ID NO:20, SEQ ID NO:22, SEQ ID NO:23, SEQ ID NO:24, SEQ ID NO:27, SEQ ID NO:30, SEQ ID NO:34, SEQ ID NO:44, SEQ ID NO:45, SEQ ID NO:50, SEQ ID NO:58, and SEQ ID NO:59.

In some embodiments, the at least one microRNA comprises 5 microRNAs.

In some embodiments, the at least one microRNA comprises 10 microRNAs.

In some embodiments, the at least one microRNA comprises 20 microRNAs.

In some embodiments, the microRNA has at least about 90% identity to a sequence selected from the group consisting of SEQ ID NO.s:01-74, and a fragment comprising at least 10 consecutive nucleotides thereof.

In some embodiments, the microRNA has at least about 95% identity to a sequence selected from the group consisting of SEQ ID NO.s:01-74, and a fragment comprising at least 10 consecutive nucleotides thereof.

In some embodiments, a two-fold change in the expression level of the microRNA is indicative of an increased metastatic potential.

In some embodiments, a five-fold change in the expression level of the microRNA is indicative of an increased metastatic potential.

In some embodiments, a ten-fold change in the expression level of the microRNA is indicative of an increased metastatic potential.

In some embodiments, the cancer comprises breast cancer.

In some embodiments, the breast cancer comprises a pre-neoblastic cancer, an adenocarcinoma or a comedocarcinoma.

In some embodiments, the subject is mammalian.

In some embodiments, the subject is human.

In some embodiments, the identity is determined using BLASTN.

Some embodiments include methods for identifying a therapeutic compound comprising: contacting a target cell with a test compound; and determining whether the test compound significantly changes the level of at least one microRNA, wherein the microRNA comprises at least about 80% sequence identity to a sequence selected from the group consisting of SEQ ID NO.s:01-74, and a fragment comprising at least 10 consecutive nucleotides thereof.

Some embodiments also include comparing the level of the microRNA in a target cell which has not been contacted with the test compound to the level of the microRNA in a target cell contacted with the test compound.

In some embodiments, the microRNA has at least about 90% identity to a sequence selected from the group consisting of SEQ ID NO.s:01-74, and a fragment comprising at least 10 consecutive nucleotides thereof.

In some embodiments, the microRNA comprises at least about 95% identity to a sequence selected from the group consisting of SEQ ID NO.s:01-74, and a fragment comprising at least 10 consecutive nucleotides thereof.

Some embodiments also include determining whether the test compound reduces the level of a microRNA selected from the group consisting of SEQ ID NO.s:01-60, and SEQ ID NO:61.

Some embodiments also include determining whether the test compound reduces the level of a microRNA selected from the group consisting of SEQ ID NO:01, SEQ ID NO:17, SEQ ID NO:19, SEQ ID NO:20, SEQ ID NO:22, SEQ ID NO:23, SEQ ID NO:24, SEQ ID NO:27, SEQ ID NO:30, SEQ ID NO:34, SEQ ID NO:44, SEQ ID NO:45, SEQ ID NO:50, SEQ ID NO:58, and SEQ ID NO:59.

Some embodiments also include determining whether the test compound increases the level of a microRNA selected from the group consisting of SEQ ID NO.s:62-73, and SEQ ID NO:74.

In some embodiments, the at least one microRNA comprises 5 microRNAs.

In some embodiments, the at least one microRNA comprises 10 microRNAs.

In some embodiments, the at least one microRNA comprises 20 microRNAs.

In some embodiments, the target cell comprises a cancer cell.

In some embodiments, the target cell comprises a breast cancer cell.

In some embodiments, the target cell is selected from a pre-neoblastic cancer cell, an adenocarcinoma cell, a comedocarcinoma cell, or a spheroid-forming cell.

In some embodiments, the target cell is mammalian.

In some embodiments, the target cell is human.

In some embodiments, the identity is determined using BLASTN.

Some embodiments include kits for evaluating the presence, absence or metastatic potential of a cancer in a subject comprising a detection reagent that binds at least one microRNA comprising 80% identity to a sequence selected from the group consisting of SEQ ID NO.s:01-74, a sequence complementary to any one of SEQ ID NO.s:01-74, and a fragment comprising at least 10 consecutive nucleotides thereof.

In some embodiments, the at least one microRNA comprises 5 microRNAs.

In some embodiments, the at least one microRNA comprises 10 microRNAs.

In some embodiments, the at least one microRNA comprises 20 microRNAs.

In some embodiments, the microRNA has at least about 90% identity to a sequence selected from the group consisting of SEQ ID NO.s:01-74, a sequence complementary to any one of SEQ ID NO.s:01-74, and a fragment comprising at least 10 consecutive nucleotides thereof.

In some embodiments, the microRNA has at least about 95% identity to a sequence selected from the group consisting of SEQ ID NO.s:01-74, a sequence complementary to any one of SEQ ID NO.s:01-74, and a fragment comprising at least 10 consecutive nucleotides thereof.

In some embodiments, the cancer comprises breast cancer.

In some embodiments, the breast cancer comprises a pre-neoblastic cancer, an adenocarcinoma, or a comedocarcinoma.

In some embodiments, the subject is mammalian.

In some embodiments, the subject is human.

Some embodiments include kits for evaluating the presence, absence or metastatic potential of a cancer in a subject comprising a detection reagent that binds at least one microRNA having a sequence selected from the group consisting of SEQ ID NO.s:01-74, a sequence complementary to any one of SEQ ID NO.s:01-74, and a fragment comprising at least 10 consecutive nucleotides thereof.

In some embodiments, the at least one microRNA comprises 5 microRNAs.

In some embodiments, the at least one microRNA comprises 10 microRNAs.

In some embodiments, the at least one microRNA comprises 20 microRNAs.

In some embodiments, the cancer comprises breast cancer.

In some embodiments, the breast cancer comprises a pre-neoblastic cancer, an adenocarcinoma, or a comedocarcinoma.

In some embodiments, the subject is mammalian.

In some embodiments, the subject is human.

Some embodiments include methods of treating breast cancer comprising administering a therapeutically effective amount of an agent which increases the expression level of Merlin protein to a subject having breast cancer.

In some embodiments, the agent is a nucleic acid encoding Merlin or fragment thereof.

In some embodiments, the agent reduces the extent of Merlin phosphorylation.

In some embodiments, the agent reduces phosphorylation of Merlin at residue Threonine 230, at residue Serine 315, or at both residues.

In some embodiments, the agent reduces the extent of Merlin ubiquitination.

In some embodiments, the agent reduces the expression level of a microRNA that targets Merlin.

In some embodiments, the microRNA is selected from the group consisting of SEQ ID NO:01, SEQ ID NO:17, SEQ ID NO:19, SEQ ID NO:20, SEQ ID NO:22, SEQ ID NO:23, SEQ ID NO:24, SEQ ID NO:27, SEQ ID NO:30, SEQ ID NO:34, SEQ ID NO:44, SEQ ID NO:45, SEQ ID NO:50, SEQ ID NO:58, and SEQ ID NO:59.

In some embodiments, the agent comprises an isolated nucleic acid selected from a small hairpin RNA (shRNA); a small interfering RNA (siRNA), a micro RNA (miRNA), an antisense polynucleotide, and a ribozyme.

In some embodiments, the subject is mammalian.

In some embodiments, the subject is human.

Some embodiments include methods of treating breast cancer comprising administering a therapeutically effective amount of an agent which decreases the expression level of a nucleic acid encoding OPN or the expression level of OPN protein to a subject having breast cancer.

In some embodiments, the agent comprises an isolated nucleic acid selected from a small hairpin RNA (shRNA), a small interfering RNA (siRNA), a micro RNA (miRNA), an antisense polynucleotide, and a ribozyme.

In some embodiments, the nucleic acid comprises a sequence encoding OPN or a fragment thereof, a sequence encoding antisense OPN or a fragment thereof, or an antisense nucleic acid complementary to a sequence encoding OPN or a fragment thereof.

In some embodiments, the subject is mammalian.

In some embodiments, the subject is human.

### BRIEF DESCRIPTION OF THE DRAWINGS

FIG. 1 depicts the inverse expression of Merlin and OPN in breast cancer tissues. FIG. 1A is a series of micrographs of normal breast tissue and invasive breast cancer stained for Merlin or OPN. Panels a-f depict normal breast tissues (a, b), and invasive breast cancer tissues (c-f) stained for Merlin. Panels g-l depict normal breast tissues (g, h), and invasive breast cancer tissues (i-l) stained for OPN. Panels a, g; b, h; c, i; d, j; e, k; and f, l are each paired serial sections. FIG. 1B is a graph of the staining intensity of Merlin in tissue sample groups characterized by grade of tumor. (†) indicates a statistically significant difference relative to normal breast tissues. FIG. 1C is a graph of the percentage of each particular tissue sample group expressing Merlin. FIG. 1D is a graph of the staining intensity of OPN in each tissue sample group characterized by grade of tumor. FIG. 1E is a graph of the percentage of each particular tissue sample group expressing OPN. FIG. 1F is a graph of the percentage of each tissue sample group characterized by grade of tumor expressing Merlin and OPN (: Merlin expression; ▪: OPN but no Merlin expression; ♦: OPN expression).

FIG. 2 depicts increased OPN transcript levels and unchanged Merlin transcript levels in breast cancer tumor tissues relative to normal breast tissue. FIGS. 2A and 2D are graphs of the relative transcript levels in normal and tumor breast tissues of Merlin or OPN, respectively. FIG. 2B and FIG. 2C are graphs of the relative transcript levels of Merlin in normal breast tissue and breast tumor tissues characterized by grade of tumor or stage of disease, respectively. FIG. 2E and FIG. 2F are graphs of the relative transcript levels of OPN in normal breast tissue and breast tumor tissues characterized by grade of tumor or stage of disease, respectively.

FIG. 3 depicts suppression of malignant behavior of breast cancer cells by Merlin. FIGS. 3A and 3B show Western blot of Merlin from SUM159 or MDA-MB-231 transfected with Merlin, respectively. FIGS. 3C and 3D show graphs of the number of foci formed by SUM159 or MDA-MB-231 transfectants, respectively. FIGS. 3E and 3F show graphs of the number of SUM159 or MDA-MB-231 transfectant cells invaded through matrigel, respectively. FIG. 3G shows a graph of the average distance migrated in a wound healing assay by SUM159 transfectants. FIG. 3H is a graph of the number of colonies formed under anchorage-independent conditions by SUM159 transfectants. FIG. 1 is a graph of mean tumor diameter in xenografts injected with SUM159 transfectant cells, tumor size is represented as mean tumor diameter (̂ p<0.0001 relative to vector controls; 4 mice were assessed per group). FIG. 3J is a graph of mean tumor diameter in xenografts injected with MDA-MB-231 transfectant cells (̂ p<0.016 relative to vector controls; 4 mice were assessed per group).

FIG. 4 depicts OPN targeting Merlin for Akt-mediated proteasomal degradation. FIG. 4A is a Western blot of SUM159 cells transfected with Merlin and treated with OPN and Lactacystin. FIG. 4B shows a Western blot of MCF10AT cells treated with OPN and Akt inhibitor IV. FIG. 4C shows a Western blot of MCF10AT treated with OPN, Lactacystin, and Akt inhibitor IV. The smear represents ubiquitinated Merlin. FIG. 4D is a Western blot of SUM159 transfected with HA-ubiquitin and Merlin and treated with OPN (100 ng/ml), Lactacystin (10 μM) and Akt inhibitor IV. FIG. 4E is a Western blot of MDA-MB-435 cells treated with the PI-3-kinase inhibitor, wortmannin and Lactacystin. Cells were pre-treated with Wortmannin (100 nM) for 1 hr followed by Lactacystin for 4.5 hours. FIG. 4F shows a Western blot of MDA-MB-435 cells transfected with HA-ubiquitin and pcDNA3-Merlin or pcDNA3-Merlin+pSuper-OPNi and treated with Lactacystin (10 μM) and Akt inhibitor IV (10 μM) for 5 hours.

FIG. 5 depicts OPN initiated signaling causes phosphorylation of Merlin at Serine 315. FIG. 5A is a Western blot of SUM159 cells transfected with Merlin and treated with OPN was probed for total Merlin and phosphorylated Merlin (Serine 315). GAPDH was used as a loading control. FIG. 5B is a Western blot of SUM159 cells were transfected with Merlin (WT) or T230A S315A Merlin mutant and treated with OPN and Lactacystin. Cell lysates were probed for total Merlin. GAPDH was used as a loading control. Mutant Merlin (T230A S315A) is not degraded in response to OPN, whereas wild-type Merlin is degraded by OPN. FIGS. 5C and FD are graphs of percent of foci formed for SUM159 cells transfected with Vector-control, wild-type Merlin and T230A S315A Merlin mutant and not treated with OPN or treated with OPN, respectively. FIG. 5E is a graph of percent colonies formed in soft agar by SUM159 cells transfected with vector-control, wild-type Merlin and T230A S315A, and not treated or treated with OPN.

FIG. 6 depicts the enhancement of tissue identification and discriminatory power of Merlin by OPN. FIG. 6A a logistic plot using Merlin as a predictor variable to distinguish between normal and tumor tissues. FIG. 6B is a logistic plot of OPN as a predictor variable and indicates that OPN by itself, is not reliably able to discriminate between normal and tumor tissues (p=0.2872; ROC area=0.6040). FIG. 6C is a ROC curve for logistic model with Merlin and OPN as predictor variables to distinguish between normal and tumor tissues and indicates that OPN does not augment the discriminatory power of Merlin (whole model test p=0.0517; ROC area=0.7234). FIG. 6D is a logistic plot using data from only the normal tissues that stained for Merlin and the entire dataset of tumor tissue staining for Merlin and indicates that Merlin has a very high discriminatory power for distinguishing between normal and tumor tissues (p<0.0001; ROC area=0.93). FIG. 6E is a logistic plot using data from only from the tumor tissues that stained for OPN and the entire dataset of normal tissue staining for Merlin and indicates that OPN has discriminatory power for distinguishing between normal and tumor tissues (p<0.0007; ROC area=0.7023). FIG. 6F is a ROC curve for logistic model utilizing non-zero Merlin values for normal tissues and non-zero OPN values for tumor tissues as predictor variables to distinguish between normal and tumor tissues and indicates that OPN augments the discriminatory power of Merlin (whole model test p<0.0001; R₂=0.81; ROC area=0.9917).

FIG. 7 is a graph of the relative expression of Merlin in various cell lines and depicts expression of exogenous Merlin relative to endogenous Merlin expressed in normal breast tissues and immortalized breast epithelial cell lines (HME and MCf10A).

FIG. 8 depicts changes in OPN mRNA expression unaccompanied by significant changes in Merlin mRNA expression. FIG. 8 (left panel) is a graph of relative expression of Merlin and OPN in Hyperplastic Enlarged Lobular Units (HELU) and Normal Terminal Duct Lobular Units (NTDLU). FIG. 8 (right panel) shows a graph of relative expression of Merlin and OPN in cases of Infiltrating Ductal Carcinoma (IDC), Infiltrating Lobular Carcinoma (ILC), Lobular control cells (LC) and Ductal control cells (DC).

FIG. 9 shows a graph of relative luciferase activity in cells co-transfected with luciferase reporter constructs containing the OPN promoter and expression constructs containing Merlin, or control expression constructs.

FIG. 10 shows a graph of relative TOPFLASH activity in cells co-transfected with TOPFLASH reporter constructs containing the β-catenin promoter and expression constructs containing Merlin, or control expression constructs.

FIG. 11 is a panel of immunocytographs of cells transfected with Merlin expression construct or a control expression construct and stained for Merlin (TRITC, red stain), β-catenin (FITC, green stain), and cell nucleus (DAPI, blue stain).

FIG. 12 is a panel of immunocytographs of cells transfected with a Merlin knockdown construct (sh Merlin) or a control knockdown construct (Vector) and stained for β-catenin (TRICT, red stain), and cell nucleus (DAPI, blue stain).

FIG. 13 is a graph of the relative change of NF-2 (Merlin) and β-catenin mRNA levels in cells transfected with either a Merlin expression construct (Merlin) or a control expression construct (Vector).

FIG. 14 depicts an interaction between Merlin and β-catenin. FIG. 14 (left panel) is a Western blot of an immunoprecipitation with Merlin and probed with β-catenin, the arrow shows a band with the estimated size of β-catenin. FIG. 14 (right panel) is a Western blot of an immunoprecipitation with β-catenin and probed with Merlin, the arrow shows a band with the estimated size of Merlin.

FIG. 15 is a series of photomicrographs of the spheroid forming cells (SFCs), MCF7-SFC, MCF10AT-SFC, DCIS-SFC, derived from the MCF7, MCF10-AT, and MCF10DCIS.com parent cell lines, respectively. FIG. 15B is a graph of mean tumor diameter over time for various numbers of DCIS-SFC cells injected into athymic nude mice.

FIG. 16 shows a Venn diagram of differentially expressed miRNAs common between the spheroid-forming cell lines DCIS-SFC, MCF7-SFC, and MCF10AT-SFC. Differential expression was relative to each spheroid-forming cell line's parent cell lines, namely, DCIS.com, MCF7, and MCF10-AT.

FIG. 17 shows a Western blot of DCIS, MCF7, and MCF10AT cells, and subpopulations of DCIS, MCF7, and MCF10AT enriched for spheroid-forming cells, probed with Merlin and β-actin.

FIGS. 18A and 18B show a series of graphs of the fold change in the level of expression of particular miRNAs in the spheroid-forming cell lines DCIS-SFC, MCF7-SFC, and MCF10AT-SFC relative to the level of each miRNA in the parent of each SFC-cell line. FIG. CA depicts the miRNAs: hsa-let-7a, hsa-let-7b, hsa-let-7c, hsa-let-7e. FIG. CB depicts the miRNA mir-361.

### DETAILED DESCRIPTION

Embodiments of the present disclosure relate to methods and compositions for the diagnosis and treatment of breast cancer. In some embodiments, the present disclosure relates to the use of Merlin, OPN and particular microRNAs for evaluating the presence, absence or metastatic potential of breast cancer in a subject and for identifying therapeutic compounds.

Unlike malignancies of the nervous system, there were no mutations identified in the tumor suppressor, Merlin (Moesin-Ezrin-Radixin-Like proteIN), in breast cancer. Indeed, while Merlin has been extensively explored in tumors arising from the nervous system, its role in breast cancer is understudied. Early studies reported that mutations in Merlin were not detected in breast cancer (19). In a separate study, Yaegashi et al. reported infrequent involvement of mutations in the NF2 gene (encoding for Merlin) in an independent cohort of 60 breast cancer patients (20). Dai et al. reported that the estrogen-response gene and tumor suppressor, NHREF, likely acts in conjunction with Merlin to transduce a growth suppressive signal (42). Thus, while there are sporadic references regarding Merlin in breast cancer, the functional and biological roles of Merlin in breast cancer have largely been ignored due to the absence of detectable mutations and the lack of reports of change at the transcript level.

Described herein is an examination of Merlin expression in breast cancer tissues using immunohistochemistry and real-time PCR. Applicants have discovered that expression of Merlin protein (assessed immunohistochemically) was significantly decreased in breast cancer tissues compared to normal tissue. Merlin transcript levels were comparable in both breast cancer tissues and normal tissues. In addition to a significant decrease in the levels of Merlin protein in breast cancer tissues, Applicants also discovered an increase in the level of expression of the tumor promoting protein, osteopontin (OPN) and nucleic acid encoding OPN in breast cancer tissue compared to normal tissue. A model using the relationship between OPN and Merlin was tested with a logistic regression model applied to immunohistochemistry data. This identified consistent decrease in immunohistochemical expression of Merlin in breast tumor tissues. Applicants also describe herein the discovery of particular microRNAs deregulated in highly tumorigenic spheroid-forming cells derived from breast cancer cell lines.

Merlin, encoded by the NF2 gene, is frequently inactivated in tumors of the nervous system (1-7). Merlin complexes with ERM (Ezrin-Radixin-Moesin) proteins that link the cytoskeleton to glycoproteins in the cell membrane (7). Merlin is critically involved in regulating cell growth and proliferation. In vitro, Merlin mediates contact inhibition and inhibits invasiveness (8,9). Underlying the tumor suppressor function of Merlin is likely a combination of the signaling pathways that attribute its ability to suppress Ras and Rac (9-11), negatively regulate FAK, downregulate expression of cyclin D1 (12), inhibit the p21-activated kinase, Pak1 (13) and interfere with the interaction between CD44 and Hyaluronan (10,14). The stability of Merlin protein is regulated, in part, by Akt-mediated phosphorylation at Threonine 230 and Serine 315 (15). Phosphorylation at these amino acids leads to Merlin degradation by ubiquitination. The reduced levels of Merlin in tumors of the nervous system are predominantly brought about by mutations or loss of heterozygosity (4, 16-18). However, Merlin's role in breast cancer has been largely ignored due to early, sporadic studies that did not detect mutations in tumor tissues (19,20).

OPN is a secreted phosphoglycoprotein (21) that acts as an effector of tumor progression and metastasis at several levels (22,23). Elevated OPN is a marker for advanced breast cancer and multiple other cancer histotypes (24-30). OPN2 initiated signaling activates NF-κB, PI-3-kinase and Akt pathways (31-33) and manifests as enhanced cell proliferation and survival, migration and adhesion (30).

Applicants describe herein that while the transcript levels of Merlin are unaltered in breast cancer tissues, there is a decrease in Merlin expression at the protein level in breast tumors, concomitant with an increase in OPN expression. The studies described herein reveal that OPN-initiated signaling induced Akt-mediated phosphorylation and degradation of Merlin in breast cancer cells. Further, restoration of Merlin in breast cancer cells functionally impeded their malignant behavior. Logistic regression consistently identified decreased Merlin staining intensity in tumor tissues. It also showed that given the Merlin intensity, OPN enhances discrimination between normal and tumor tissue. Thus, the availability of Merlin in breast tumors is likely regulated at the post-translational level. This is unexpected as Merlin was not found to be mutated or compromised at the transcript level in breast cancers.

In the present application, it is demonstrated that the level of Merlin transcript does not appreciably change in breast tumor tissues. Thus, it was intriguing to note a significant decrease in the immunohistochemical staining for Merlin, suggestive of the fact that Merlin protein expression is decreased in breast cancer. In contrast, the oncoprotein, OPN showed an increase in expression at the transcript levels as well as at the protein level. OPN binding to cell surface receptors, such as the integrins, cause several signal transduction pathways to turn on culminating in enhanced proliferation, migration and survival (22). The studies described herein demonstrate that OPN induces Akt-mediated phosphorylation of Merlin. This phosphorylation targets Merlin for ubiquitin-mediated degradation in breast cancer cells resulting in decreased overall cellular pools of endogenous Merlin.

Ubiquitin-mediated degradation of tumor suppressors such as p53, PML, PTEN and VHL has also been documented to be responsible for the decreased availability of the respective proteins in tumor cells (43,44). As described herein degradation of endogenous Merlin is one of the ways by which OPN-initiated signaling removes the check of this tumor suppressor. OPN is a secreted protein, hence it is available to the tumor cells in their microenvironment. Given this fact, the implications of the findings described herein can have important considerations for understanding and appreciating the effects that OPN can have on tumor cells. OPN levels increase during pathogenesis of breast cancer. OPN is also available to the tumor cells from the surrounding stromal and inflammatory cells that infiltrate the tumor. OPN-initiated signaling via Akt results in phosphorylation of Merlin and its subsequent degradation. Being a secreted protein that utilizes a variety of receptors, OPN can influence signaling in surrounding tumor cells causing a reduction in Merlin protein levels as a ‘bystander effect’ resulting in a widespread degradation induced decrease in Merlin. As such, while OPN has been reported to induce ubiquitin-mediated degradation of Stat1 (45), the present application reports that OPN causes degradation of a tumor suppressor protein.

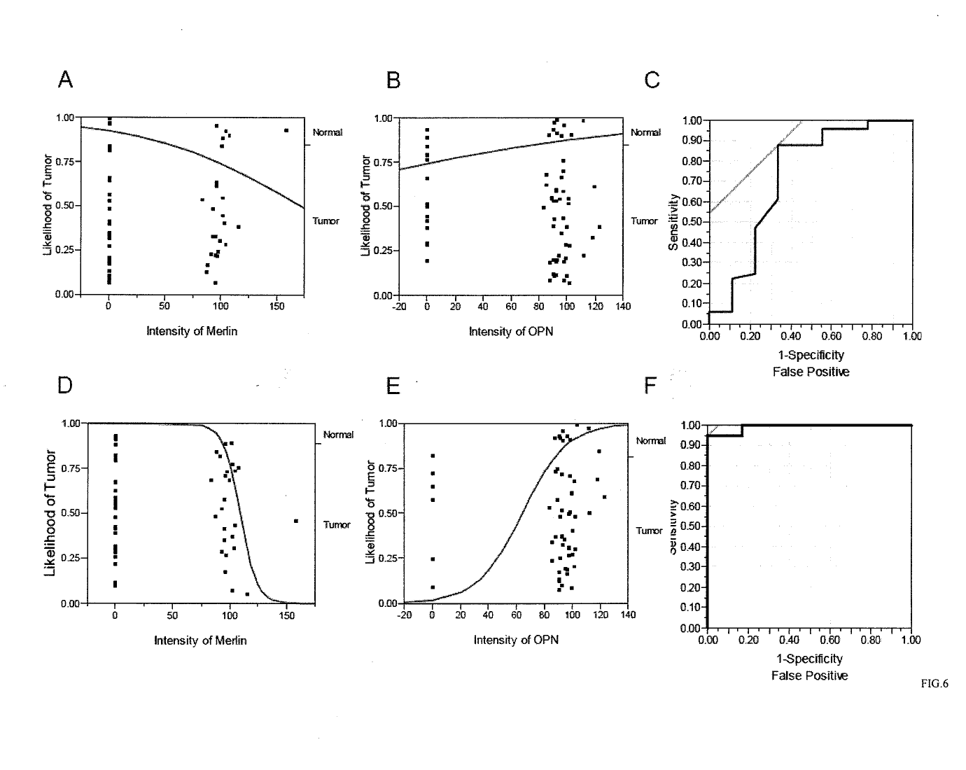

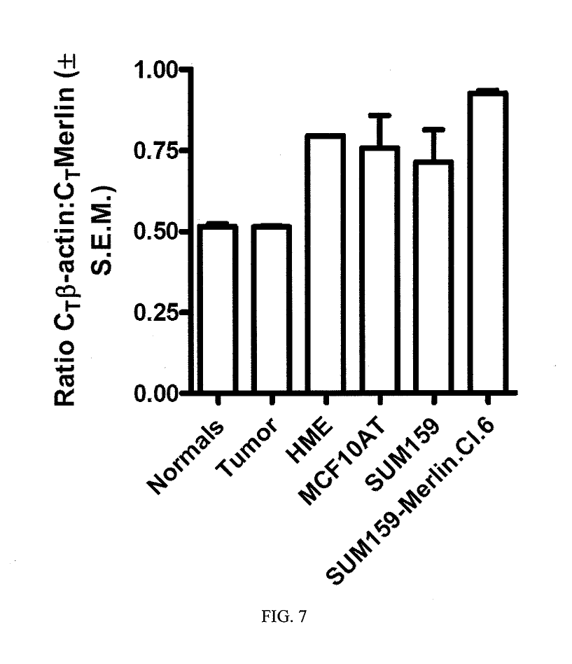

Although in breast cancer Merlin may not be a prototypic tumor suppressor gene that conforms to the classic definition of Knudsen's two-hit hypothesis, the present application demonstrates that Merlin has a tumor suppressor activity in breast cancer. Restoration of Merlin in breast tumor cells less than 2-fold upregulated relative to normal tissues, functionally blunted their malignant properties (FIG. 7). As such, the inverse relationship between Merlin and OPN that was observed in clinical specimens is diagnostically useful. Logistic regression identified Merlin intensity as a good predictor for immunohistochemical identification of tumor tissue. It also showed that enhanced staining intensity of OPN enhances tissue identification, when combined with the staining intensity of Merlin in breast tumor tissues. The significance of Merlin expression and its function in breast cancer had been ignored thus far. The present application demonstrates a functional role for Merlin in breast cancer and is also the first report of OPN in causing the degradation of a tumor suppressor protein. Thus, the present application elucidates the utility of Merlin and OPN as important biomarkers in breast cancer and also identify a novel mechanism for the decrease in Merlin expression in breast cancer.

### β-Catenin

β-catenin, a key factor in the Wnt signaling pathway, has essential functions in the regulation of cell growth and differentiation. Aberrant β-catenin signaling has been linked to various disease pathologies, including an important role in tumorigenesis.

β-catenin, has a dual function in epithelial cells. It acts in E-cadherin-mediated cell-cell adhesions and instigates Wnt-induced gene programs in the nucleus (Clevers H. Cell. 2006; 127:2-7). Signaling events in the Wnt/β-catenin cascade revolve around the regulation of the non-membrane-bound pool of β-catenin with potential to act in transcription. Without a Wnt signal, uncomplexed β-catenin in the cytosol is rapidly phosphorylated by a multi-protein complex composed of the scaffolding proteins Axin and Adenomatous Polyposis Coli (APC) and the kinases CK1 and GSKβ. Phospho-β-catenin is immediately recognized and degraded through the ubiquitin-proteasome system. Thus, the Axin-APC complex keeps cytosolic levels of β-catenin low. Simultaneous binding of Wnt to Frizzled (Fz) and Lrp5/6 coreceptors leads, via recruitment of the cytoplasmic effector protein Dishevelled (Dvl), to inhibition of the Axin/APC protein complex (MacDonald B T, et al., Wnt/beta-catenin signaling: components, mechanisms and diseases. Dev Cell. 2009; 17:9-26). As a consequence the levels of cytoplasmic β-catenin rise, followed by its transport into the nucleus and the activation of target gene transcription by β-catenin/TCF complexes (Stadeli R, et al., Curr Biol. 2006; 16:378-385). Inappropriate elevation of β-catenin levels and uncontrolled activation of target genes is linked-to a multitude of human disorders, including cancer and neurodegenerative diseases (Clevers H. Cell. 2006; 127:2-7; and MacDonald B T, et al., Dev Cell. 2009; 17:9-26).

MicroRNAs (miRNAs)

Some embodiments of the methods and compositions provided herein relate to the use of particular microRNAs (miRNAs) to diagnose the presence, absence, or metastatic potential of cancer. miRNAs are short RNAs, on average only 22 nucleotides long processed from longer precursor miRNAs. miRNAs include post-transcriptional regulators that bind to complementary sequences on target mRNAs, usually resulting in translational repression and gene silencing. As such, miRNAs are members of the class of non-coding RNAs that have emerged as regulators of gene expression. They have been reported to regulate gene expression at the level of both transcription and translation (Nelson K M, et al. Mol Cancer Ther. 2008; 7: 3655-60, incorporated herein by reference in its entirety). Their role in cancer pathogenesis has become increasingly evident. Several recent studies have identified miRNAs as novel diagnostic and prognostic indicators and therapeutic targets (Takamizawa J, et al. Cancer Res. 2004; 64: 3753-6; Wu W, et al. Int J Cancer. 2007; 120: 953-60; Jay C, et al. DNA Cell Biol. 2007; 26: 293-300; Cho WC. Mol Cancer. 2007; 6: 60; Negrini M, Calin G A. Breast Cancer Res. 2008; 10: 203; and Lowery A J, et al. Clin Cancer Res. 2008; 14: 360-5, each incorporated herein by reference in its entirety). Recent evidence indicates that miRNAs can function as tumor suppressors or oncogenes (Zhang B, et al. Dev Biol. 2007; 302:1-12, incorporated herein by reference in its entirety). Oncogenic miRNAs (oncomiRs) are miRNAs with a defined role in cancer. In clinically derived breast cancer specimens the expression of several miRNAs was deregulated in correlation with certain pathologic features (Iorio M V, et al. Cancer Res. 2005; 65: 7065-70, incorporated herein by reference in its entirety). Specifically, miRNAs have been reported to influence processes such as epithelial-to-mesenchymal transition (Gregory P A, et al. Nat Cell Biol. 2008; 10: 593-601, incorporated herein by reference in its entirety) and tumor invasion and metastasis (Tavazoie S F, et al. Nature. 2008; 451: 147-52; Huang Q, et al. Nat Cell Biol. 2008; 10: 202-10; Ma L, et al., Nature. 2007; 449: 682-8, each incorporated herein by reference in its entirety). miRNAs have also been implicated in tamoxifen resistance of breast cancer (Zhao J J, et al. J Biol Chem. 2008; 283: 31079-86; Miller T E, et al. J Biol Chem. 2008; 283: 29897-903, each incorporated herein by reference in its entirety) and doxorubicin resistance of breast cancer (Kovalchuk O, et al. Mol Cancer Ther. 2008; 7: 2152-9, incorporated herein by reference in its entirety). Some embodiments provided herein relate to the use of microRNAs for evaluating the presence of a cancer, or the metastatic potential of a cancer or tumor in a subject. More embodiments relate to the use of microRNAs for identifying therapeutic agents. More embodiments relate to kits for evaluating the presence of a cancer in a subject including reagents for the detection of certain microRNAs.

### Methods for Diagnosis and Prognosis

Some embodiments of the methods and compositions provided herein relate to evaluating the presence or metastatic potential of a cancer, such as breast cancer, in a sample. As used herein, “sample” can include a biological sample, such as a tissue sample. The sample can be an in vivo sample, ex vivo sample, in vitro sample. Some embodiments include evaluating the presence or metastatic potential of a cancer, such as breast cancer, from a subject. As used herein, “subject” can include an animal, such as a mammal, such as a human. In some embodiments, the sample comprises a sample removed from the subject's body, and expression levels of protein and/or nucleic acids can be measured ex vivo, namely, outside the subject's body.

In some embodiments, the expression level of a biomarker in a sample can be measured. Examples of biomarkers include Merlin, such as Merlin protein, phosphorylated Merlin protein (e.g., at residues Threonine 230, and Serine 315), OPN, such as a nucleic acid encoding OPN, or OPN protein, and nucleic acids with a particular level of sequence identity to microRNAs provided herein, such as SEQ ID NO.s:01-74. In some embodiments, a nucleic acid can have a level of identity with a nucleic acid provided herein, such as SEQ ID NO.s:01-74 of at least about 50%, 60%, 70%, 80%, 90%, 95%, 99%, and 100%. Methods to measure the level of a protein or nucleic acid in a sample are well known in the art and examples are also described herein. The level of identity between sequences, such as nucleic acid sequences or protein sequences, can be a relationship between two or more sequences, as determined by comparing the sequences. A number of algorithms (which are generally computer implemented) for comparing the sequences are widely available, or can be produced by one of skill. These methods include, e.g., the local homology algorithm of Smith and Waterman (1981) Adv. Appl. Math. 2:482; the homology alignment algorithm of Needleman and Wunsch (1970) J. Mol. Biol. 48:443; the search for similarity method of Pearson and Lipman (1988) Proc. Natl. Acad. Sci. (USA) 85:2444; and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Software for performing sequence identity (and sequence similarity) analysis using the BLAST algorithm is described in Altschul et al. (1990) J. Mol. Biol. 215:403-410. This software is publicly available, e.g., through the National Center for Biotechnology Information at <ncbi.nlm.nih.gov>. In some embodiments, sequence identity can be determined using BLAST. In some embodiments, the default parameters of each of the foregoing algorithms or software can be utilized in determining the level of sequence identity.

In some embodiments, the expression level of a biomarker in a test sample can be compared to the expression level of the biomarker in normal tissue, or cancerous tissue with a known metastatic potential. The normal tissue, or cancerous tissue with a known metastatic potential can be obtained from the same subject as the test sample, different individuals, or a plurality of individuals. In some embodiments, the test sample and normal tissue, or cancerous tissue with a known metastatic potential can be obtained at the same time, or with a period in between. Alternatively, in some embodiments, the expression level of a biomarker in a test sample can be compared to a level which has been previously determined to be indicative of normal tissue or of a particular metastatic potential.

The change in the level of expression of a biomarker can be used to determine the presence, absence or metastatic potential of a cancer in a sample. For example, the decrease in the level of expression of Merlin protein in a test sample relative to the level of expression of Merlin protein in a normal tissue can indicate the presence or metastatic potential of a breast cancer. In some embodiments, the relative decrease in the level of expression of Merlin protein in a test sample can be at least about 5%, 10%, 20%, 30%, 40%, 50%, 60%, 70%, 80%, 90%, 100%, and more.

In some embodiments, the increase in the level of expression of phosphorylated Merlin protein (e.g., Merlin protein phosphorylated at residue Threonine 230, at residue Serine 315, or both) in a test sample relative to the level of expression of phosphorylated Merlin protein in a normal tissue can indicate the presence or metastatic potential of a breast cancer. In some embodiments, the relative increase in the level of expression of phosphorylated Merlin protein in a test sample can be at least about 5%, 10%, 20%, 30%, 40%, 50%, 60%, 70%, 80%, 90%, 100%, and more.

In some embodiments, the increase in the level of expression of a nucleic acid encoding OPN or the increase in the level of expression of a OPN protein in a test sample relative to the level of expression of a nucleic acid encoding OPN or the increase in the level of expression of a OPN protein in a normal tissue can indicate the presence or metastatic potential of a breast cancer. In some embodiments, the relative increase in the level of expression of a nucleic acid encoding OPN or the increase in the level of expression of a OPN protein in a test sample can be at least about 5%, 10%, 20%, 30%, 40%, 50%, 60%, 70%, 80%, 90%, 100%, and more.

In some embodiments, the decrease in the level of expression of Merlin protein in a test sample relative to the level of expression of Merlin protein in a normal tissue, and the increase in the level of expression of a nucleic acid encoding OPN or the increase in the level of expression of a OPN protein in a test sample relative to the level of expression of a nucleic acid encoding OPN or the level of expression of a OPN protein in a normal tissue can indicate the presence of a breast cancer. In some embodiments, the ratio of the relative decrease in the expression level of Merlin protein expression in a test sample to the relative increase in the expression level of a nucleic acid encoding OPN or the relative increase in the level of expression of a OPN protein in a test sample, each with respect to the expression level in normal tissue, can indicate the presence of a breast cancer. Examples of ratios for the decrease in the relative level of Merlin expression to increase in the relative level of OPN expression include: at least about 100:1, 50:1, 20:1, 10:1, 5:1, 2:1, 1:1, 1:2, 1:5, 1:10, 1:20, 1:50, and 1:100.

In more embodiments, the change in the level of expression of a microRNA in a test sample relative to the level of expression of the microRNA in a normal tissue can indicate the presence or metastatic potential of a breast cancer. The relative change may be any change which is statistically significant. In some embodiments, the relative change in the level of expression of a microRNA protein in a test sample can be at least about 30%, 40%, 50%, 60%, 70%, 80%, 90%, 100%, 150%, 200%, and more.

### Methods of Detecting Biomarkers

Expression levels such as levels of nucleic acids such as microRNA and mRNA, levels of protein, and levels of biological activity of a protein or mRNA can be measured by various methods.

For example, measurement of protein levels, such as levels of Merlin protein or OPN protein, may utilize binding agents. There are a variety of assay formats known to those of ordinary skill in the art for using a binding agent to detect protein markers in a sample. See, e.g., Harlow and Lane, Antibodies: A Laboratory Manual, Cold Spring Harbor Laboratory, 1988. In general, the presence, absence, or metastatic potential of a cancer in a subject may be determined by (a) contacting a biological sample obtained from a subject with a binding agent; (b) determining the level of the polypeptide that binds to the binding agent; and (c) comparing the level of polypeptide with a predetermined cut-off value indicative of the presence, absence or metastatic potential of the cancer.

In a preferred embodiment, an assay involves the use of binding agent immobilized on a solid support to bind to the polypeptide in the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. In such embodiments, the binding agent can comprise an antibody or fragment thereof specific to Merlin or OPN. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length breast tumor proteins, such as Merlin protein or OPN protein, and polypeptide portions thereof to which the binding agent binds.

The solid support may be any material known to those of ordinary skill in the art to which the binding agent may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In some embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art may be used, such as bovine serum albumin or TWEEN 20. (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with breast cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% TWEEN 20. The second antibody, which contains a reporter group, may then be added to the solid support. Reporter groups are well known in the art.

The detection reagent is then incubated with the immobilized antibody-polypeptide complex for an amount of time sufficient to detect the bound detection reagent. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

In some embodiments, to determine the presence, absence, or metastatic potential of a cancer, such as breast cancer, the signal detected from the reporter group that remains bound to the solid support is generally compared to a signal that corresponds to a predetermined cut-off value indicative to the presence, absence, or metastatic potential of a cancer. In one embodiment, the cut-off value is the average mean signal obtained when an immobilized antibody is incubated with samples from patients without the cancer. In general, a sample generating a signal that is three standard deviations away from the predetermined cut-off value is considered positive for the cancer. For example, a reduced level of Merlin protein or an increased level of OPN protein may be indicative of the presence of cancer, or the metastatic potential of cancer, such as breast cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer. It will be understood that this method can also be applied in situations where a decrease in the level of expression of a marker is used to detect cancer, or indicate the metastatic potential of cancer.

In another embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described herein. In the strip test format, one end of the membrane to which binding agent is bound is immersed in a solution containing the sample. The sample migrates along the membrane through a region containing second binding agent and to the area of immobilized binding agent. The amount of immobilized antibody indicates the presence, absence, stage, or metastatic potential of a cancer. Typically, the concentration of second binding agent at that site generates a pattern, such as a line, that can be read visually. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

In some embodiments, the level of phosphorylated proteins, such as phosphorylated Merlin, can be measured. In some methods, phosphorylated protein isoforms can be distinguished from unphosphorylated protein isoforms. Methods to detect phosphorylated proteins and unphosphorylated proteins are well known in the art. In some embodiments, an antibody specific to a phosphorylated protein isoform can be used to determine the presence of the phosphorylated protein isoform, and to measure the relative level of the phosphorylated protein isoform in a sample. See e.g., U.S. Patent App No. 20100008901, incorporated by reference herein in its entirety.

Of course, numerous other assay protocols exist that are suitable for use with the markers, such as the protein markers, described herein. The above descriptions are intended to be examples only. It will be apparent to those of ordinary skill in the art that the above protocols may be readily modified to use marker polypeptides to detect antibodies that bind to such polypeptides in a biological sample. The detection of such marker-specific antibodies may correlate with the presence of a cancer.

As noted herein, a cancer, the stage of cancer, or metastatic potential of cancer, may also, or alternatively, be detected based on the level of mRNA encoding OPN. For example, at least two oligonucleotide primers may be employed in a polymerase chain reaction (PCR) based assay to amplify a portion of a marker cDNA derived from a biological sample, wherein at least one of the oligonucleotide primers is specific for a polynucleotide encoding the marker. The amplified cDNA is then separated and detected using techniques well known in the art, such as gel electrophoresis. Similarly, oligonucleotide probes that specifically hybridize to a polynucleotide encoding a tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding a marker described herein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence as disclosed herein. Techniques for both PCR based assays and hybridization assays are well known in the art (see, e.g., Mullis et al., Cold Spring Harbor Symp. Quant. Biol., 51:263, 1987; Erlich ed., PCR Technology, Stockton Press, NY, 1989).

One embodiment employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater change in expression in several dilutions of the test patient sample as compared to the same dilutions of the non-cancerous sample may typically considered positive.

In some embodiments, microRNAs can be identified and/or quantified. The level of a microRNA in a sample can be measured using any technique that is suitable for detecting RNA expression levels in a biological sample. Suitable techniques for determining RNA expression levels in biological sample include amplification-based and hybridization-based assays. Such techniques are also useful to determine the level of a nucleic acid encoding OPN in a cell.

Amplification-based assays include quantitative amplification in which the amount of amplification product will be proportional to the amount of template in the original sample. Methods of real-time quantitative PCR or RT-PCR using TaqMan probes are well known in the art and are described in for example, Heid et al. 1996, Real time quantitative PCR, Genome Res., 10:986-994; and Gibson et al., 1996, A novel method for real time quantitative RT-PCR, Genome Res. 10:995-1001. A quantitative real-time RT-PCR method that can determine the expression level of the nucleic acid transcripts is described in Jiang, J., et al. (2005), Nucleic Acids Res. 33, 5394-5403; Schmittgen T. D., et al. (2004), Nucleic Acids Res. 32, E43; and U.S. Provisional Application Ser. No. 60/656,109, filed Feb. 24, 2005, the entire contents of which are incorporated herein by reference. Other examples of amplification-based assays for detection of microRNAs are well known in the art, see for example the description in US PAT Appl. No. 2006/0078924, the entire contents of which are incorporated herein by reference. Hybridization-based assays can also be used to detect the level of microRNAs in a sample. These assays, including for example Northern blot analysis, in-situ hybridization, solution hybridization, and RNAse protection assay (Ma Y J, et al. (1996) RNase protection assay, Methods, 10:273-8) are well known to those of skill in the art.

A suitable technique for determining the level of RNA, such as microRNAs or messenger RNAs, in a biological sample is Northern blotting. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

In addition to Northern and other RNA hybridization techniques, determining the levels of RNA transcripts, such as microRNAs or messenger RNAs, can be accomplished using the technique of in situ hybridization. This technique requires fewer cells than the Northern blotting technique, and involves depositing whole cells onto a microscope cover slip or slide and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference. Probes for measuring RNA transcripts and miRNAs can include probes comprising at least about 50%, 60%, 70%, 80%, 85%, 90%, 95%, 99%, or 100% sequence identity to a sequence that includes any one of SEQ ID NO:01-73, and SEQ ID NO:74. In some embodiments, probe can have at least about 50%, 60%, 70%, 80%, 85%, 90%, 95%, 99%, or 100% sequence identity to the sequence complementary to one of SEQ ID NO.s:01-74, or to at least about 10, 15, 20, 25 consecutive nucleotides complementary to one of SEQ ID NO.s:01-74.

### Methods for Identifying Therapeutic Agents

Some of the methods and compositions provided herein relate to identifying a therapeutic agent. As used herein “therapeutic agent” includes a compound useful for preventing or treating a physiological condition, such as a disease, such as cancer. Therapeutic compounds can include any compound, for example, small molecules, proteins, and nucleic acids.

In some embodiments for identifying a therapeutic agent, a target cell is contacted with a test compound. In some embodiments, the target cell comprises a cancer cell, such as a breast cancer cell, an IDC cell, a distant metastasis cell, a pre-neoblastic cancer cell, an adenocarcinoma cell, a comedocarcinoma cell, or a spheroid-forming cell. In some embodiments, the target cell is mammalian, such as human. The expression level of a biomarker, such as Merlin protein, phosphorylated Merlin protein (e.g., Merlin protein phosphorylated at residue Threonine 230, at residue Serine 315, or both), a nucleic acid encoding OPN, OPN protein, or microRNAs provided herein, such as SEQ ID NO.s:01-74, can be measured.

In some embodiments for identifying a therapeutic agent, the expression level of a biomarker in a target cell contacted with a test compound is compared to the expression level of the biomarker in a cell not contacted with the test compound. Some such embodiments can also include determining whether the level of the biomarker in the target cell contacted with the test compound is changed significantly relative to the level of the biomarker in a cell not contacted with the test compound. As used herein, “significantly” can refer to a change of at least about 5%, 10%, 20%, 30%, 40%, 50%, 60%, 70%, 80%, 90%, 100%, or more.

In some embodiments, the expression level of Merlin protein in a target cell contacted with a test compound relative to the expression level of Merlin protein in a target cell not contacted with the test compound can increase by at least about 5%, 10%, 20%, 30%, 40%, 50%, 60%, 70%, 80%, 90%, 100%, or more. In some embodiments, the extent of phosphorylation of Merlin protein can decrease by at least about 5%, 10%, 20%, 30%, 40%, 50%, 60%, 70%, 80%, 90%, 100%, or more. In some embodiments, the expression level of a nucleic acid encoding OPN or the expression level of OPN protein in a target cell contacted with a test compound relative to the expression level of a nucleic acid encoding OPN or the expression level of OPN protein in a target cell not contacted with the test compound can decrease by at least about 5%, 10%, 20%, 30%, 40%, 50%, 60%, 70%, 80%, 90%, 100%, or more.

In some embodiments for identifying a therapeutic agent, the expression level of at least one microRNA in a target cell contacted with a test compound relative to the expression level of the at least one microRNA in a target cell not contacted with the test compound can change by at least about 5%, 10%, 20%, 30%, 40%, 50%, 60%, 70%, 80%, 90%, 100%, or more. In more embodiments, the relative level of the at least one microRNA increases. In some such embodiments, the microRNA can have a particular level of sequence identity to at least one sequence including SEQ ID NO.s:62-74. In more embodiments, the relative level of the at least one microRNA can decrease. In some such embodiments, the microRNA can have a particular level of sequence identity to at least one sequence including SEQ ID NO.s:01-61. In some embodiments, the microRNA can have a particular level of sequence identity with a nucleic acid provided herein, such as SEQ ID NO.s:01-74. In each of the foregoing embodiments, the level of sequence identity may be at least about 50%, 60%, 70%, 80%, 90%, 95%, 99%, and 100%. In some embodiments, the level of sequence identity can be determined using BLAST, e.g., BLASTN with default parameters. In some embodiments, the levels of a plurality of microRNAs can be measured in a target cell contacted with a test compound, such as at least 3 microRNAs, at least 5 microRNAs, at least 10 microRNAs, at least 15 microRNAs, at least 20 microRNAs, at least 25 microRNAs, and more.

### Methods of Treatment

Some embodiments of the compositions and methods provided herein relate to the prevention or treatment of diseases and disorders, such as breast cancer. In some embodiments, a therapeutically effective amount of an agent can be administered to a subject. In some embodiments, therapeutic agents can be identified using methods described herein.

In some embodiments, the agent increases the expression level of Merlin, such as Merlin protein, in a cell, such as an increase of at least about 10%, at least about 20%, at least about 30%, at least about 40%, at least about 50%, at least about 60%, at least about 70%, at least about 80%, at least about 90%, and at least about 100%. In some embodiments, the agent reduces the extent of total Merlin phosphorylation in a cell, such as by at least about 10%, at least about 20%, at least about 30%, at least about 40%, at least about 50%, at least about 60%, at least about 70%, at least about 80%, at least about 90%, and at least about 100%. In some embodiments, the agent reduces the extent of Merlin protein phosphorylation at residue Threonine 230, at residue Serine 315, or at both residues. In some embodiments, the agent reduces the extent of Merlin protein ubiquitination in a cell, such as a reduction in at least about 10%, at least about 20%, at least about 30%, at least about 40%, at least about 50%, at least about 60%, at least about 70%, at least about 80%, at least about 90%, and at least about 100%. In some embodiments, the agent reduces the expression level of a microRNA that targets Merlin, such as reduction at least about 10%, at least about 20%, at least about 30%, at least about 40%, at least about 50%, at least about 60%, at least about 70%, at least about 80%, at least about 90%, and at least about 100%. Examples of such microRNAs include SEQ ID NO:01, SEQ ID NO:17, SEQ ID NO:19, SEQ ID NO:20, SEQ ID NO:22, SEQ ID NO:23, SEQ ID NO:24, SEQ ID NO:27, SEQ ID NO:30, SEQ ID NO:34, SEQ ID NO:44, SEQ ID NO:45, SEQ ID NO:50, SEQ ID NO:58, and SEQ ID NO:59. In some embodiments, the agent decreases the expression level of a nucleic acid encoding OPN or the expression level of OPN protein in a cell, such as a reduction in the expression level of a nucleic acid encoding OPN or the expression level of OPN protein of at least about 10%, at least about 20%, at least about 30%, at least about 40%, at least about 50%, at least about 60%, at least about 70%, at least about 80%, at least about 90%, and at least about 100%.

In some embodiments, the levels of Merlin protein can be increased by contacting a cell with a nucleic acid encoding Merlin (e.g., SEQ ID NO:75) or with a fragment of at least 10, 20, 50, and 100 consecutive nucleotides thereof. Methods to deliver such nucleic acids to the cell of a subject are well known and examples are described herein.

In some embodiments, the levels of an OPN protein, nucleic acid encoding an OPN protein, or microRNA targeting Merlin can be reduced using RNA interference or antisense technologies. RNA interference is an efficient process whereby double-stranded RNA (dsRNA), also referred to herein as siRNAs (small interfering RNAs) or ds siRNAs (double-stranded small interfering RNAs), induces the sequence-specific degradation of targeted mRNA in animal or plant cells (Hutvagner, G. et al. (2002) Curr. Opin. Genet. Dev. 12:225-232); Sharp, P. A. (2001) Genes Dev. 15:485-490, incorporated by reference herein in its entirety).

In mammalian cells, RNA interference can be triggered by various molecules, including 21-nucleotide duplexes of siRNA (Chiu, Y.-L. et al. (2002) Mol. Cell. 10:549-561. Clackson, T. et al. (1991) Nature 352:624-628; Elbashir, S. M. et al. (2001) Nature 411:494-498), or by micro-RNAs (miRNA), functional small-hairpin RNA (shRNA), or other dsRNAs which can be expressed in vivo using DNA templates with RNA polymerase III promoters (Zheng, B. J. (2004) Antivir. Ther. 9:365-374; Paddison, P. J. et al. (2002) Genes Dev. 16:948-958; Lee, N. S. et al. (2002) Nature Biotechnol. 20:500-505; Paul, C. P. et al. (2002) Nature Biotechnol. 20:505-508; Tuschl, T. (2002) Nature Biotechnol. 20:446-448; Yu, J.-Y. et al. (2002) Proc. Natl. Acad. Sci. USA 99(9):6047-6052; McManus, M. T. et al. (2002) RNA 8:842-850; Sui, G. et al. (2002) Proc. Natl. Acad. Sci. USA 99(6):5515-5520, each of which are incorporated herein by reference in their entirety). The scientific literature is replete with reports of endogenous and exogenous gene expression silencing using siRNA, highlighting their therapeutic potential (Gupta, S. et al. (2004) PNAS 101:1927-1932; Takaku, H. (2004) Antivir Chem. Chemother 15:57-65; Pardridge, W. M. (2004) Expert Opin. Biol. Ther. 4(7):1103-1113; Shen, W.-G. (2004) Chin. Med. J. (Engl) 117:1084-1091; Fuchs, U. et al. (2004) Curr. Mol. Med. 4:507-517; Wadhwa, R. et al. (2004) Mutat. Res. 567:71-84; Ichim, T. E. et al. (2004) Am. J. Transplant 4:1227-1236; Jana, S. et al. (2004) Appl. Microbiol. Biotechnol. 65:649-657; Ryther, R. C. C. et al. (2005) Gene Ther. 12:5-11; Chae, S-S. et al. (2004) J. Clin. Invest 114:1082-1089; de Fougerolles, A. et al. (2005) Methods Enzymol. 392:278-296, each of which is incorporated herein by reference in its entirety). Some nucleic acid molecules or constructs provided herein include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region, such as in the mRNA of OPN, and the other strand is identical or substantially identical to the first strand. An example method for designing dsRNA molecules is provided in the pSUPER RNAi SYSTEM™ (OligoEngine, Seattle, Wash.). More example methods are provided in Taxman D. J. et al. (2006) BMC Biotechnol. 6:7; and McIntyre G. J. et al. (2006) BMC Biotechnol. 6:1, each of which is incorporated by reference in its entirety.

Synthetic siRNAs can be delivered to cells by methods known in the art, including cationic liposome transfection and electroporation. siRNAs generally show short term persistence of the silencing effect (4 to 5 days in cultured cells), which may be beneficial in certain embodiments. To obtain longer term suppression of expression for targeted genes, such as OPN, and to facilitate delivery under certain circumstances, one or more siRNA duplexes, e.g., ds siRNA, can be expressed within cells from recombinant DNA constructs. Such methods for expressing siRNA duplexes within cells from recombinant DNA constructs to allow longer-term target gene suppression in cells are known in the art, including mammalian Pol III promoter systems (e.g., H1 or U6/snRNA promoter systems (Tuschl, T. (2002) Nature Biotechnol. 20:446-448) capable of expressing functional double-stranded siRNAs; (Lee, N. S. et al. (2002) Nature Biotechnol. 20:500-505; Miyagishi, M. and Taira, K. (2002) Nature Biotechnol. 20:497-500; Paul, C. P. et al. (2002) Nature Biotechnol. 20:505-508; Yu, J.-Y. et al. (2002) Proc. Natl. Acad. Sci. USA 99(9):6047-6052; Sui, G. et al. (2002) Proc. Natl. Acad. Sci. USA 99(6):5515-5520).

Nucleic acids provided herein can include microRNA which can regulate gene expression at the post transcriptional or translational level. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zheng, B. J. (2004) Antivir. Ther. 9:365-374). When expressed by DNA vectors containing polymerase III promoters, microRNA designed hairpins can silence gene expression, such as OPN expression.

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002) Nature Biotechnol. 20(10):1006-10). In vitro infection of cells by such recombinant adenoviruses allows for diminished endogenous target gene expression. Injection of recombinant adenovirus vectors into transgenic mice expressing the target genes of the siRNA results in in vivo reduction of target gene expression. In an animal model, whole-embryo electroporation can efficiently deliver synthetic siRNA into post-implantation mouse embryos (Calegari, F. et al. (2002) Proc. Natl. Acad. Sci. USA 99(22):14236-40). In adult mice, efficient delivery of siRNA can be accomplished by the “high-pressure” delivery technique, a rapid injection (within 5 seconds) of a large volume of siRNA containing solution into animal via the tail vein (Lewis, D. L. (2002) Nature Genetics 32:107-108). Nanoparticles, liposomes and other cationic lipid molecules can also be used to deliver siRNA into animals. A gel-based agarose/liposome/siRNA formulation is also available (Jiamg, M. et al. (2004) Oligonucleotides 14(4):239-48).

Nucleic acids provided herein can include an antisense nucleic acid sequence selected such that it is complementary to the entirety of OPN, a microRNA, or to a portion of OPN or a microRNA. In some embodiments, a portion can refer to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, and at least about 80%, at least about 85%, at least about 90%, at least about 95%. In some embodiments, a portion can refer up to 100%.

In some embodiments, a nucleic acid having activity to reduce OPN protein expression, to reduce the level of a nucleic acid encoding OPN, to reduce the level of a microRNA, or to increase Merlin, in a cell of a subject is further operably linked to a regulatory sequence. Regulatory sequences include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990), the disclosure of which is incorporated herein by reference in its entirety. Regulatory sequences include those which direct constitutive expression of a nucleotide sequence in many types of host cell and those which direct expression of the nucleotide sequence only in certain host cells (e.g., tissue-specific regulatory sequences). Tissue specific promoters may be used to effect transcription in specific tissues or cells so as to reduce potential toxicity or undesirable effects to non-targeted tissues. Examples include: adipose tissue: lipoprotein lipase, adipsin, acetyl-CoA carboxylase, glycerophosphate dehydrogenase, adipocyte P2; and mammary: MMTV, and whey acidic protein (WAP).

In certain embodiments, it may be desirable to activate transcription at specific times after administration of a vector comprising a nucleic acid having activity to reduce OPN protein expression, to reduce the level of a nucleic acid encoding OPN, to reduce the level of a microRNA, or to increase the expression level of Merlin, in a cell. This may be done with such promoters as those that may be regulated by hormone or cytokine. For example, in a gonadal tissue where specific steroids are produced or routed to, use of androgen or estrogen regulated promoters may be advantageous. Such promoters that are hormone regulatable include MMTV, MT-1, ecdysone and RuBisco. Other hormone regulated promoters such as those responsive to thyroid, pituitary and adrenal hormones are expected to be useful with the nucleic acids described herein. Cytokine and inflammatory protein responsive promoters that could be used include K and T Kininogen, c-fos, TNF-α, C-reactive protein, haptoglobin, serum amyloid A2, C/EBP α, IL-1, IL-6, Complement C3, IL-8, α-1 acid glycoprotein, α-1 antitrypsin, lipoprotein lipase, angiotensinogen, fibrinogen, c-jun (inducible by phorbol esters, TNF α, UV radiation, retinoic acid, and hydrogen peroxide), collagenase (induced by phorbol esters and retinoic acid), metallothionein (heavy metal and glucocorticoid inducible), Stromelysin (inducible by phorbol ester, interleukin-1 and EGF), α-2 macroglobulin and α-I antichymotrypsin. It is envisioned that any of the promoters described herein, alone or in combination with another, may be useful depending on the action desired.

Nucleic acid constructs having activity to reduce OPN protein expression, to reduce the level of a nucleic acid encoding OPN, to reduce the level of a microRNA, or to increase the expression level of Merlin, in a cell and described herein can be introduced in vivo as naked DNA plasmids, for example, using transfection, electroporation (e.g., transcutaneous electroporation), microinjection, transduction, cell fusion, DEAE dextran, calcium phosphate precipitation, use of a gene gun, or use of a DNA vector transporter (Wu et al. J. Biol. Chem., 267:963-967, 1992; Wu and Wu J. Biol. Chem., 263:14621-14624, 1988; and Williams et al. Proc. Natl. Acad. Sci. USA 88:2726-2730, 1991). A needleless delivery device, such as a BIOJECTOR® needleless injection device can be utilized to introduce nucleic acid constructs in vivo. Receptor-mediated DNA delivery approaches can also be used (Curiel et al. Hum. Gene Ther., 3:147-154, 1992; and Wu and Wu, J. Biol. Chem., 262:4429-4432, 1987). Methods for formulating and administering naked DNA to mammalian muscle tissue are disclosed in U.S. Pat. Nos. 5,580,859 and 5,589,466, both of which are herein incorporated by reference in their entireties. Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931), the disclosures of which are incorporated herein by reference in their entireties.

Alternatively, electroporation can be utilized conveniently to introduce nucleic acid constructs, having activity to reduce OPN protein expression, to reduce the level of a nucleic acid encoding OPN, to reduce the level of a microRNA, or to increase the expression level of Merlin, in a cell and described herein, into cells. Electroporation is well known by those of ordinary skill in the art (see, for example: Lohr et al. Cancer Res. 61:3281-3284, 2001; Nakano et al. Hum Gene Ther. 12:1289-1297, 2001; Kim et al. Gene Ther. 10:1216-1224, 2003; Dean et al. Gene Ther. 10:1608-1615, 2003; and Young et al. Gene Ther 10:1465-1470, 2003). For example, in electroporation, a high concentration of vector DNA is added to a suspension of host cell (such as isolated autologous peripheral blood or bone marrow cells) and the mixture shocked with an electrical field. Transcutaneous electroporation can be utilized in animals and humans to introduce heterologous nucleic acids into cells of solid tissues (such as muscle) in vivo. Typically, the nucleic acid constructs are introduced into tissues in vivo by introducing a solution containing the DNA into a target tissue, for example, using a needle or trochar in conjunction with electrodes for delivering one or more electrical pulses. For example, a series of electrical pulses can be utilized to optimize transfection, for example, between 3 and ten pulses of 100 V and 50 msec. In some cases, multiple sessions or administrations are performed.

Another well known method that can be used to introduce nucleic acid constructs, having activity to reduce OPN protein expression, to reduce the level of a nucleic acid encoding OPN, to reduce the level of a microRNA, or to increase the expression level of Merlin, in a cell and described herein, into host cells is biolistic transformation. One method of biolistic transformation involves propelling inert or biologically active particles at cells, e.g., U.S. Pat. Nos. 4,945,050, 5,036,006; and 5,100,792, the disclosures of which are hereby incorporated by reference in their entireties. Generally, this procedure involves propelling inert or biologically active particles at the cells under conditions effective to penetrate the outer surface of the cell and to be incorporated within the interior thereof. When inert particles are utilized, the plasmid can be introduced into the cell by coating the particles with the plasmid containing the exogenous DNA. Alternatively, the target cell can be surrounded by the plasmid so that the plasmid is carried into the cell by the wake of the particle.

Alternatively, nucleic acid constructs, having activity to reduce OPN protein expression, to reduce the level of a nucleic acid encoding OPN, to reduce the level of a microRNA, or to increase the expression level of Merlin, in a cell and described herein, can be introduced in vivo by lipofection. Synthetic cationic lipids designed to limit the difficulties and dangers encountered with liposome mediated transfection can be used to prepare liposomes for in vivo transfection of a gene encoding a marker (Feigner et al. Proc. Natl. Acad. Sci. USA 84:7413-7417, 1987; Mackey, et al. Proc. Natl. Acad. Sci. USA 85:8027-8031, 1988; Ulmer et al. Science 259:1745-1748, 1993, the disclosures of which are incorporated herein by reference in their entireties). The use of cationic lipids can promote encapsulation of negatively charged nucleic acids, and also promote fusion with negatively charged cell membranes (Feigner and Ringold Science 337:387-388, 1989, the disclosure of which is incorporated by reference herein in its entirety). Particularly useful lipid compounds and compositions for transfer of nucleic acids are described in WO95/18863 and WO96/17823, and in U.S. Pat. No. 5,459,127, incorporated herein by reference in their entireties.

In some embodiments, the nucleic acid constructs, having activity to reduce OPN protein expression, to reduce the level of a nucleic acid encoding OPN, to reduce the level of a microRNA, or to increase the expression level of Merlin, in a cell and described herein, are viral vectors. Methods for constructing and using viral vectors are known in the art (See e.g., Miller and Rosman, BioTech., 7:980-990, 1992). Preferably, the viral vectors are replication defective, that is, they are unable to replicate autonomously in the target cell. In some cases, the replication defective virus retains the sequences of its genome that are necessary for encapsulating the viral particles. DNA viral vectors commonly include attenuated or defective DNA viruses, including, but not limited to, herpes simplex virus (HSV), papillomavirus, Epstein Barr virus (EBV), adenovirus, adeno-associated virus (AAV), Moloney leukemia virus (MLV) and human immunodeficiency virus (HIV) and the like. Defective viruses, that entirely or almost entirely lack viral genes, are preferred, as defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al. Mol. Cell. Neurosci., 2:320-330, 1991, the disclosure of which is incorporated herein by reference in its entirety), defective herpes virus vector lacking a glycoprotein L gene (See for example, Patent Publication RD 371005 A, incorporated herein by reference in its entirety), or other defective herpes virus vectors (See e.g., WO 94/21807; and WO 92/05263, incorporated herein by reference in their entireties); an attenuated adenovirus vector, such as the vector described by Stratford-Perricaudet et al. (J. Clin. Invest., 90:626-630 1992; La Salle et al., Science 259:988-990, 1993, the disclosure of which is incorporated herein by reference in its entirety); and a defective adeno-associated virus vector (Samulski et al., J. Virol., 61:3096-3101, 1987; Samulski et al., J. Virol., 63:3822-3828, 1989; and Lebkowski et al., Mol. Cell. Biol., 8:3988-3996, 1988, the disclosures of which are incorporated herein by reference in their entireties).

In some embodiments, the viral vectors, having activity to reduce OPN protein expression, to reduce the level of a nucleic acid encoding OPN, to reduce the level of a microRNA, or to increase the expression level of Merlin, in a cell and described herein, may be adenovirus vectors. Adenoviruses are eukaryotic DNA viruses that can be modified to efficiently deliver a nucleic acid of the disclosure to a variety of cell types. Various serotypes of adenovirus exist. Of these serotypes, preference is given, within the scope of the present disclosure, to type 2, type 5 or type 26 human adenoviruses (Ad 2 or Ad 5), or adenoviruses of animal origin (See e.g., WO94/26914 and WO2006/020071, the disclosures of which are incorporated herein by reference in their entireties). Those adenoviruses of animal origin that can be used within the scope of the present disclosure include adenoviruses of canine, bovine, murine (e.g., Mav1, Beard et al. Virol., 75-81, 1990, the disclosure of which is incorporated herein by reference in its entirety), ovine, porcine, avian, and simian (e.g., SAV) origin. In some embodiments, the adenovirus of animal origin is a canine adenovirus, such as a CAV2 adenovirus (e.g. Manhattan or A26/61 strain (ATCC VR-800)). More examples of methods for treating a cell in a subject can be found in International Application No. PCT/US2011/029093, incorporated herein by reference in its entirety.

Some embodiments include pharmaceutical compositions comprising a nucleic acid which reduces OPN protein expression, reduces the level of a nucleic acid encoding OPN, reduces the level of a microRNA, or increases the expression level of Merlin, in a cell, and a suitable carrier. While any suitable carrier known to those of ordinary skill in the art may be employed in the pharmaceutical compositions described herein, the type of carrier will typically vary depending on the mode of administration. Compositions described herein may be formulated for any appropriate manner of administration, including for example, topical, oral, nasal, mucosal, intravenous, intracranial, intraperitoneal, subcutaneous and intramuscular administration. Carriers for use within such pharmaceutical compositions are biocompatible, and may also be biodegradable. In certain embodiments, the formulation preferably provides a relatively constant level of active component release.

### Indications

Embodiments of the methods and compositions provided herein relate to cancers, such as breast cancer. Breast cancers include ductal carcinomas and lobular carcinomas. Ductal carcinomas include invasive/infiltrating ductal carcinoma (IDC), and ductal carcinoma in situ (DCIS). Breast cancers can be classified by histopathology, grade, stage, and receptor status. Grade of a breast cancer refers to the appearance of the cells relative to normal breast tissue; cancerous cells are less differentiated. Low grade cancerous cells include well differentiated cells, intermediate grade cancerous cells include moderately differentiated cells, and high grade cancerous cells include poorly differentiated cells. Stage of a breast cancer is based on the size of a tumor, whether the tumor has spread to a lymph node in the arm pits, and whether the tumor has metastasized. Stage 0 is a pre-cancerous or marker condition and may include DCIS or lobular carcinomas in situ (LCIS). Stage 1-3 includes tumors within the breast or regional lymph nodes. Stage 4 includes metastatic tumors. Breast cancer cells may or may not have surface markers such as estrogen receptors (ER), progesterone receptors (PR), or HER2/neu. Distant metastasis includes breast cancer cells that settle and colonize specific sites of a body.

### Kits

Some embodiments of the methods and compositions provided herein relate to kits for evaluating the presence or metastatic potential of a breast cancer in a subject. Such kits can include one or more components such as reagents for performing an assay, reagents for preserving a sample, and the like, instruments for collecting a sample, instruments for performing an assay, vessels for storing reagents, vessels for storing a sample, and the like, and instructions for use of the kit.

In some embodiments, kits provided herein include a detection reagent that binds to Merlin protein or which assess the phosphorylation state of the Merlin protein. In more embodiments, a kit can include a detection reagent that binds to phosphorylated Merlin protein (e.g., Merlin protein phosphorylated at residue Threonine 230, Serine 315, or both), or a nucleic acid encoding OPN or OPN protein. Some embodiments include a kit for evaluating the presence or metastatic potential of a breast cancer such as an infiltrating ductal carcinoma (IDC), or a distant metastasis.

In some embodiments, kits provided herein include a detection reagent that binds at least one microRNA comprising at least about 50%, 60%, 70%, 80%, 85%, 90%, 95%, 99%, or 100% sequence identity to a sequence that includes any one of SEQ ID NO:01-73, and SEQ ID NO:74. In some embodiments, the reagent can be a nucleic acid having at least about 50%, 60%, 70%, 80%, 85%, 90%, 95%, 99%, or 100% sequence identity to the sequence complementary to one of SEQ ID NO.s:01-74, or to at least about 10, 15, 20, 25 consecutive nucleotides complementary to one of SEQ ID NO.s:01-74. In some embodiments, a kit can include more reagents to detect at least 1, 5, 10, or 20 microRNAs. The reagent to detect the microRNA can detect a microRNA with at least about 50%, 60%, 70%, 80%, 90%, 95%, 99%, 100% identity to a sequence that includes any one of SEQ ID NO:01-73, and SEQ ID NO:74. Sequence identity may be determined by a variety of methods described herein, for example, using BLASTN with default parameters. Some embodiments include a kit for evaluating the presence or metastatic potential of a breast cancer such as a pre-neoblastic cancer, an adenocarcinoma, or a comedocarcinoma.

While the present invention has been described in some detail for purposes of clarity and understanding, one skilled in the art will appreciate that various changes in form and detail can be made without departing from the true scope of the invention.

### EXAMPLES

### Example 1

### Materials and Methods

Cell Culture—MCF10AT, MDA-MB-231 and MDA-MB-435 cells were cultured as previously described (34). SUM159 cells were grown in DMEM/F-12 supplemented with FBS, insulin, and hydrocortisone in a humidified 5% CO2 environment. The lineage infidelity of MDAMB-435 cells has been discussed in several papers (35-37). The MDAMB-435 cell line as a model due to the fact that it naturally expresses copious OPN. Stable Merlin-expressing transfectants of MDA-MB-231 and SUM 159 cells were generated by transfecting a Merlin-expressing construct. Empty-vector was transfected as control; stable transfectants were selected on G418 (Invitrogen, Carlsbad, Calif.). MCF10DCIS.com cell lines were grown in DMEM/F-12 (Invitrogen, Carlsbad, USA) supplemented with 5% heat inactivated horse serum (Invitrogen), 100 ng/ml cholera toxin (Calbiochem San Diego Calif.), 10 μg/ml insulin (Sigma, St. Louis, Mo.), 25 ng/ml EGF (Sigma, St. Louis, Mo.), and 500 ng/ml hydrocortisone (Sigma). The MCF10DCIS.com cell line is locally aggressive and was obtained by serial xenograft passages of the premalignant, tumorigenic MCF10AT cells in SCID mice. The MCF7 cells were grown in DMEM/F-12 (Invitrogen) supplemented with 5% heat inactivated horse serum (Invitrogen) and 10 μg/ml insulin (Sigma). The spheroid-forming cell population (SFC) from MCF10AT, MCF7 and MCF10DCIS.com cells was enriched by culturing them under conditions of compromised adherence in low attachment tissue culture plates (Corning, Corning, N.Y.) in DMEM-F12 (Invitrogen) supplemented with 0.4% BSA (Sigma), 25 ng/ml EGF (Sigma) and 10 ng/ml bFGF (Sigma).

Western Blotting Analysis—Immunoblotting was done with anti-Merlin (Santa Cruz Biotech, Santa Cruz, Calif.), phospho-Ser473-Akt (Cell Signaling, Danvers, MAT), total Akt (Cell Signaling), anti-mouse HA (Santa Cruz), antiphospho-Ser315 Merlin, and anti-GAPDH (Cell Signaling). Anti-rabbit or anti-mouse HRP conjugated secondary antibody was used for detection and blots were developed with SuperSignal substrate (Pierce, Rockford, Ill.) and exposed using a Fuji LAS3000 imager.

Transfection and Drug Treatment—Cells were transfected with empty vector, Merlin (WT; wild-type) or T230A S315A Merlin mutant and treated with clasto-Lactacystin β-Lactone (Sigma, St. Louis, Mo.) for 2 hours. Recombinant OPN (100 ng/mL) (R&D Systems, Minneapolis, Minn.) was added and cells were lysed after 6 hours. Where indicated, cells were first treated with Akt inhibitor IV (Calbiochem) in serum free media for 30 min followed by 100 ng/mL human rOPN for 24 hours.

Immunoprecipitation—Cells were transfected with pcDNA3.1 HA-ubiquitin alone or in combination with pIRES2-myc-Merlin and incubated for 24 hrs. Cells were treated with 10 μM Lactacystin, 100 ng/ml OPN and 10 μM AKT inhibitor IV for 12 hrs and lysed in NP-40 buffer. The lysate was immunoprecipitated with anti-Merlin antibody and the immunoprecipitate was assessed by immunoblotting.

microRNA analysis —RNA quality was assessed using the Bioanalyser2100 (Agilent, Palo Alto, Calif., USA) and RNA measurement on the Nanodrop instrument (Wilmington, Del., USA). The samples were labeled using the miRCURY™ Hy3™/Hy5™ labeling kit and hybridized on the miRCURY™ LNA Array (v. 8.1) (Exiqon, Denmark).

Real-time quantitative PCR of tissue array—TissueScan plates (Origene, Rockville, Md.) were assessed for the expression of OPN and Merlin transcripts using the manufacturer's protocol. The reaction was carried out in a Bio-Rad iCycler iQ5 using the following program: activation step of 50° C. for 2 minutes, then 42 cycles of 95° C. for 5 minutes, 95° C. for 15 seconds, and 60° C. for 1 minute. Data was expressed as fold change (2^{-ΔΔCT}). Statistical analysis was conducted using JMP version 7.0.1 (SAS, Inc., Cary, N.C.). A 5% level of significance was used to determine significance of results. The data were summarized using mean, standard deviation, and standard error of mean. The Pearson's correlation coefficient was used to determine correlation between numerical variables such as age. Wilcoxon test was used to compare CRTR levels of Merlin and OPN by group (normal or tumor), grade, and stage. A p-value of <0.05 was considered significant between groups.

Analysis of miRNA levels by real-time RT-PCR—cDNA was generated using the MicroRNA Reverse Transcription kit (Applied Biosystems). Total RNA (200 ng) was used to synthesize cDNA using primers specific to either U6 (control) or the miRNAs being assessed. PCR was done using cDNA with either U6 or miRNA-specific TaqMan primer probe sets with 1× TaqMan Universal PCR Master Mix, No AmpErase UNG (Applied Biosystems). The following thermocycling conditions were used: an initial step of 95° C. for 10 minutes followed by 40 cycles of 95° C. for 15 seconds and 60° C. for 1 minute. miRNA levels were normalized to U6 (ΔCt=Ct_{miRNA}−Ct_{U6}) levels which was used to calculate changes in miRNA (ΔΔCt). To compare changes in expression between monolayer-derived adherent breast cancer cells and SFC cells, the adherent breast cancer cells were set as calibrator which were defined as 100% and compared to their respective SFC cells. The levels of miRNA was determined as 2^{-ΔΔCt}×100% wherein ΔΔCt=ΔCt_{SFC}−ΔCt_{adherent}.

Soft agar colonization assay—Cells were seeded in soft agar in triplicate in a 6-well plate, allowed to grow for 2-3 weeks, stained with crystal violet solution. Colonies with >50 cells were microscopically counted.

Foci formation assay—Cells were transfected with empty vector or pcDNA3.1-Merlin or pcDNA3.1-T230A S315A-Merlin, detached and re-seeded in media containing selection antibiotics. Foci formed were counted after 10-14 days.

Xenograft studies—Cells (1 million) suspended in HBSS (Invitrogen) were injected into the exposed third mammary fat pad of female athymic nude mice. Orthogonal tumor measurements were recorded twice-weekly. Mean tumor diameter was calculated as the square-root of the product of orthogonal measurements.

Tumor growth assay—Cells at 70-90% confluence were detached with Trypsin-EDTA (Invitrogen), washed with chilled CMF-DPBS, and resuspended in ice-cold Hank's Balanced Salt Solution (Invitrogen) and injected into the third mammary fat pad of 6 week old, female athymic mice (Harlan Sprague-Dawley, Indianapolis, Ind., USA). The SFC cells were mechanically dissociated, counted and similarly injected into mice. Tumor size was measured weekly and mean tumor diameter calculated by taking the square root of the product of orthogonal measurements. Mice were euthanized after the mean tumor diameter reached 1.0 cm.

Immunohistochemistry—Breast tumor tissue microarrays from NCI Cooperative Breast Cancer Tissue Resource were immunohistochemically stained for OPN (AKm2A1; Santa Cruz) and Merlin (A-19; Santa Cruz) using the streptavidin biotin complex method. Staining intensity was quantitated with computer-assisted image analysis in a Dako ACIS III Image Analysis System (Glostrup, Denmark). Table 1 summarizes clinicopathological features of tissues from breast cancer patients.

TABLE 1

| Feature | | Number of patients |
|---|---|---|
| Age (years) mean ± S.D. (range) | | 60.34 ± 13.25 (31-84) |
| Ethnicity | African American | 8 |
| | Caucasian | 78 |
| Tumor size (cm) mean ± S.D. (range) | | 2.64 ± 1.52 (1.0-9.5) |
| Normal | | 9 |
| DCIS | | 9 |
| Node negative | | 25 |
| Node positive | | 26 |
| Distant metastasis | | 24 |
| ER status | Negative | 27 |
| | Positive | 64 |
| | N/A | 2 |
| PR status | Negative | 46 |
| | Positive | 45 |
| | N/A | 2 |
| Grade | 1 | 16 |
| | 2 | 36 |
| | 3 | 21 |
| T-status | T1 | 34 |
| | T2 | 34 |
| | T3 | 3 |
| | T4 | 4 |
| | TIS | 3 |
| N-status | N0 | 31 |
| | N1 | 35 |
| | N2 | 2 |
| | N3 | 1 |
| | NX | 9 |
| M-status | M0 | 54 |
| | M1 | 24 |

Statistical Analyses—Associations between intensities of Merlin and OPN expressions and patient's clinicopathologic data were assessed using the Wilcoxon rank test for categorical data and the Pearson's correlation coefficient for numerical data. The percentages of normal and tumor tissues expressing Merlin or OPN were compared using a Chi-square test. The significance of percentages of samples expressing Merlin or OPN as compared to the chance occurrence was determined using the exact binomial test. The univariate and multiple logistic regression models were fit to a binary variable normal versus tumor with Merlin and OPN as possible predictors. The possibility of developing a model using the relationship between OPN and Merlin was tested with a logistic regression model on a selected cohort of the data, scoring only the positive staining events from normal tissues for Merlin and the positive staining events from tumor tissue for OPN. The selection criteria were based on the fact that Merlin is a tumor suppressor, with a strong expression in normal tissue, whereas OPN—a tumor promoting protein, is known to be overexpressed in tumor tissue. The Chi-square test was used to assess the usefulness of model for prediction of likelihood of tumor. The effect likelihood ratio test was used to assess the usefulness of predictor variables in the model. The area under the ROC curve was used to determine the predictive ability of models and in model selection. All statistical analyses were performed using software JMP v 7 (SAS Inc.). All results with p-value<0.05 were considered statistically significant.

Statistical analyses of in vitro data—Statistical differences between groups were assessed using the Mann-Whitney test, t-test or ANOVA, using GraphPad Prism 4 software. Statistical significance was determined if the analysis reached 95% confidence.

Example nucleic acid sequence and protein sequence for human Merlin include SEQ ID NO:75 and SEQ ID NO:76, respectively. Example nucleic acid sequence and protein sequence for human OPN include SEQ ID NO:77 and SEQ ID NO:78, respectively.

### Example 2

### Merlin and OPN are Inversely Expressed in Breast Cancer Tissues

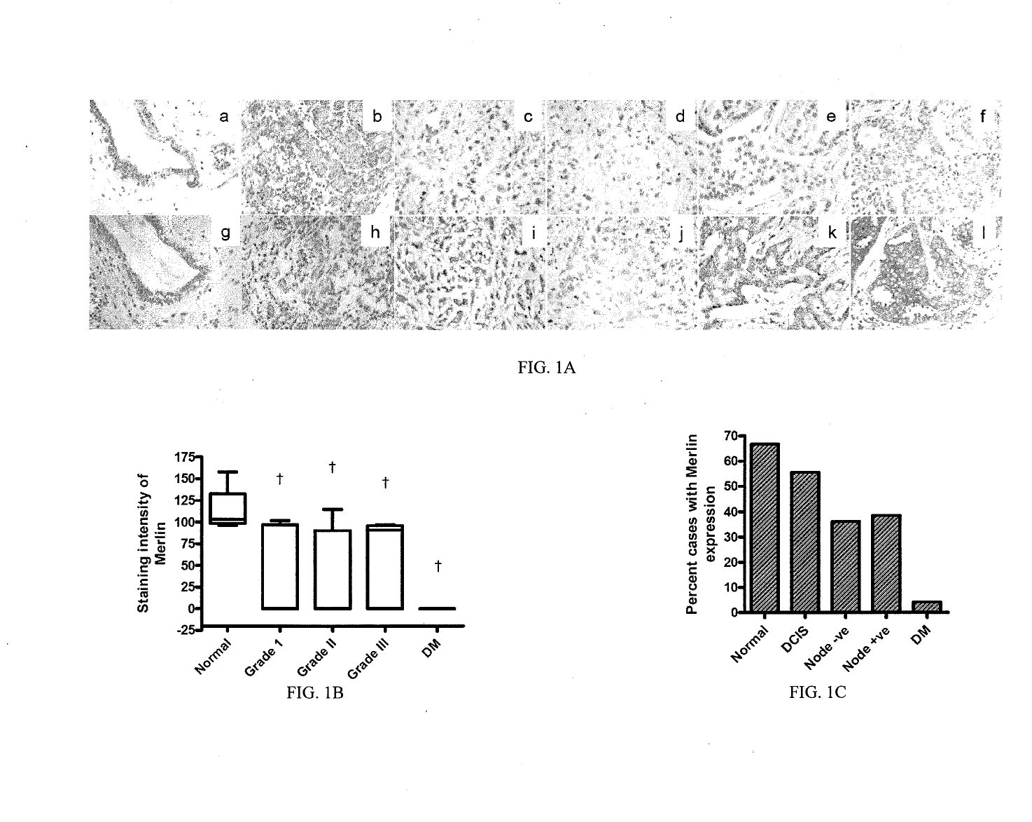

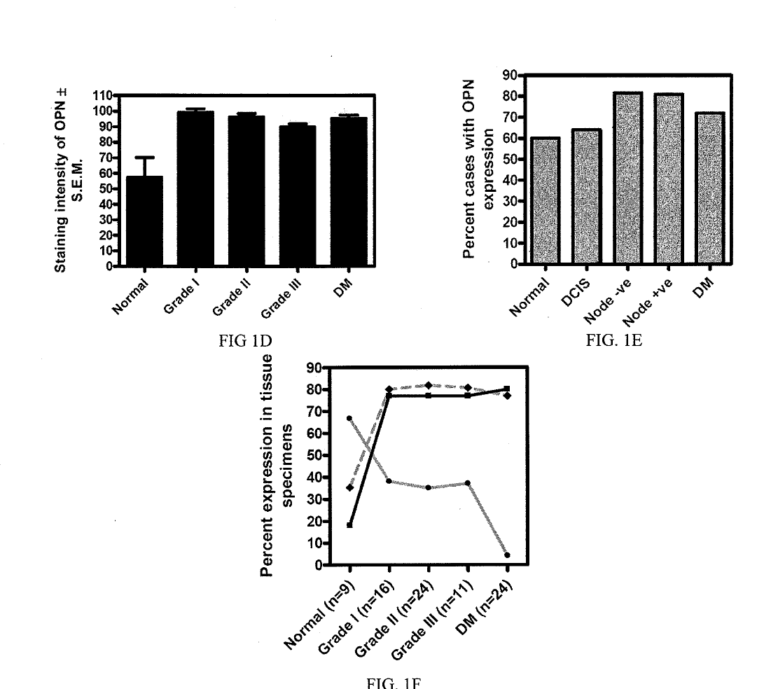

Immunohistochemical staining was performed for Merlin and OPN on serial sections from nine normal breast tissue samples and seventy-five samples of invasive breast cancer, namely, infiltrating ductal carcinoma (IDC) grades I, II, III. Notably, a decrease or loss in Merlin expression was recorded in 75% (fifty-six samples) of invasive breast cancer samples (p=0.0000097). The expression of Merlin did not change significantly with respect to ethnicity, age, ER or PR status or tumor size. FIG. 1A depicts representative photomicrographs of the results.

Relative to normal breast tissue, Merlin expression was statistically significantly lower in grade I (p=0.0026), grade II (p=0.0005), grade III (p=0.0017) tumors and in tumors with distant metastasis (p≦0.0001). Table 2 and FIG. 1B summarize the mean staining intensity for Merlin and the relative stating intensity for Merlin with respect to grade of tumor, respectively.

TABLE 2

| | | Mean staining | Standard error |
|---|---|---|---|
| Tissue | Number | intensity for | of the mean |
| characteristic | (n) | Merlin | (S.E.M.) |
| Normal | 9 | 74.2 | 19.5 |
| Grade I | 13 | 37.3 | 13.6 |
| Grade II | 25 | 27.8 | 9.1 |
| Grade III | 11 | 59.2 | 14.2 |
| Distant metastasis | 24 | 3.9 | 3.9 |

A greater proportion of normal breast tissues expressed Merlin relative to breast cancer tissues (node-negative and node-positive) and tumors with distant metastasis (p=0.0005). Relative to normal breast tissue, the level of Merlin was statistically significantly lower in node-negative (p=0.0171) and node-positive (p=0.0457) tumors and in tumors with distant metastasis (p<0.0001). The levels of Merlin in ductal carcinoma in situ (DCIS) tissues were not significantly different from normal tissue (p=0.2026). Thus, the expression of Merlin in IDC cells was observed to be significantly lower, regardless of nodal involvement. Table 3 and FIG. 1C summarize the mean staining intensity for Merlin and the distribution of samples with Merlin expression with respect to nodal involvement, respectively.

TABLE 3

| | | Mean staining | Standard error |
|---|---|---|---|
| Tissue | Number | intensity for | of the mean |
| characteristic | (n) | Merlin | (S.E.M.) |
| Normal | 9 | 74.2 | 19.5 |
| DCIS | 9 | 54.0 | 17.2 |
| Node −ve | 25 | 33.7 | 9.2 |
| Node +ve | 26 | 38.0 | 9.6 |
| Distant metastasis | 24 | 3.9 | 3.9 |

Of the fifty-six tissue, samples with a decrease or loss in Merlin expression, forty-three (77%) showed concomitant increased OPN expression. In particular, the staining intensity of OPN was observed to be significantly increased (p<0.0001) in breast cancer tissues (grades I, II, III) and in tumors showing distant metastasis (DM) relative to normal breast tissue (FIGS. 1A, panels i-l (tumor), g-h (normal); FIG. 1D). Relative to normal tissue, a greater proportion of node-negative and node-positive breast cancer tissues, and tissues showing distant metastasis relative to normal breast tissues were observed to express OPN (FIG. 1E).

In 77% of all grade I-III and distant metastasis tissue samples in which no Merlin staining was observed (53 samples), an increase in OPN staining was observed (43 samples) (FIG. 1F; p=0.000031). In twenty-three of twenty-four primary tumor samples with distant metastasis, no Merlin staining was observed (FIG. 1F; p=0.000001). Of these twenty-three primary tumor samples with distant metastasis and with no observed Merlin staining, twenty cases (80%) showed increased OPN staining (FIG. 1F; p=0.00077) In sum, Merlin protein expression is reduced or lost in invasive breast cancer and decrease or loss of Merlin expression is accompanied by an increased expression of OPN.

### Example 3

### Transcript Levels of Merlin and OPN in Breast Cancer Tissues

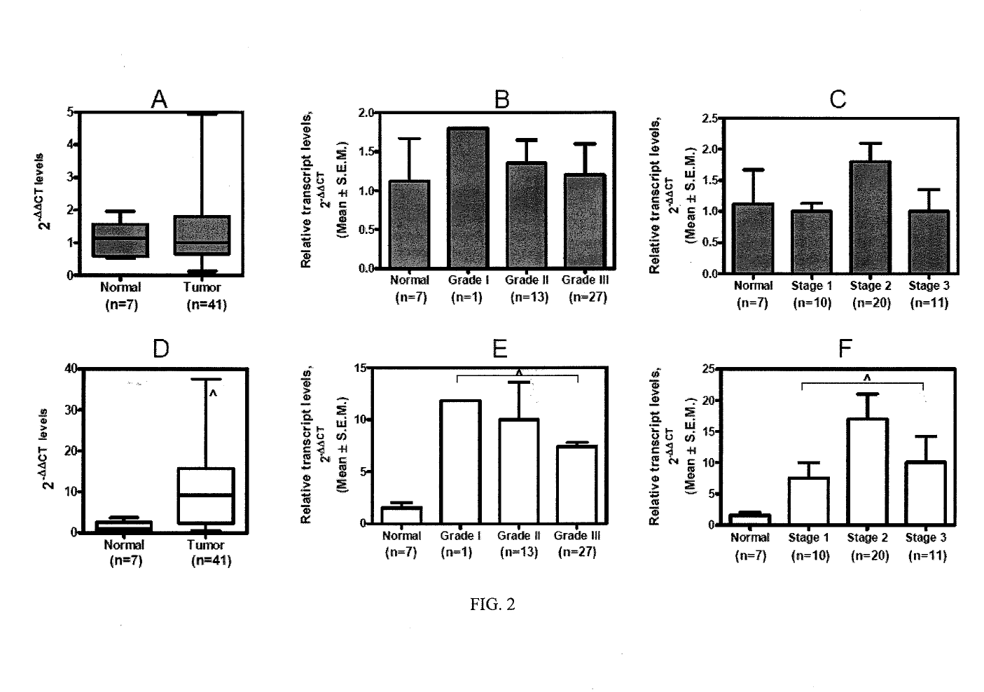

The expression of Merlin in breast tumor tissues was examined at two levels: amount of the transcript and the extent of protein expression. The transcript levels in tissues from forty-one breast cancer patients and seven normal control tissues were assessed. The transcript levels of Merlin did not show any appreciable changes (p>0.05) between normal and breast tumor derived tissues (FIG. 2A); there was also no change in the Merlin transcript levels across the different grades of tumors (p=0.6) or the disease stage (p=0.15). (FIGS. 2B, 2C). In contrast, the transcript levels of OPN were significantly (p<0.01) greater in the breast tumor tissues relative to normal tissues (FIG. 2D). The OPN transcript levels also increased significantly in tissues derived from grades II and III tumors (FIG. 2E; p=0.04) and with progression of the disease stage (FIG. 2F; p=0.01). In sum, transcript levels of Merlin in breast cancer tissues were observed to be unaltered while those of OPN were increased.

### Example 4

### Merlin Suppresses Malignant Behavior of Breast Cancer Cells

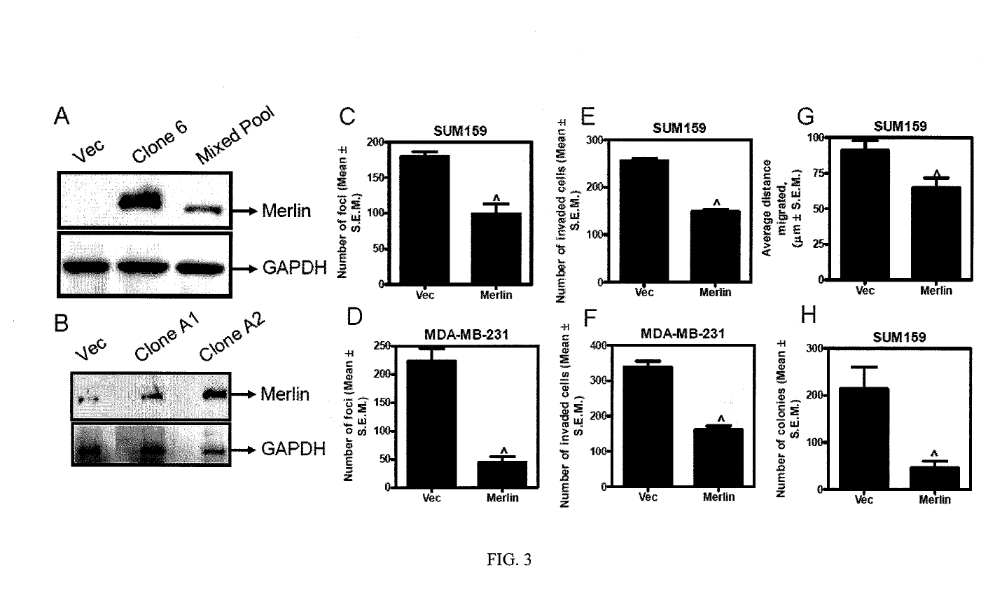

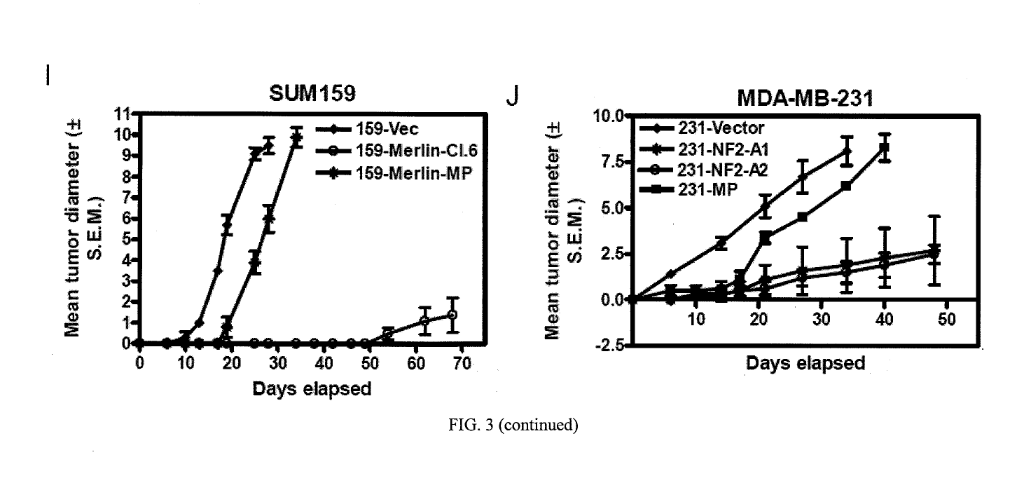

Merlin's role as a tumor suppressor is characterized in tumors of the nervous system. To examine the impact of Merlin in the malignant behavior of breast cancer cells, stable Merlin expressing transfectants were derived from the human breast cancer cell lines, SUM159 and MDA-MB-231 (FIGS. 3A, 3B). Expression of Merlin caused a significant reduction in the ability of SUM159 (̂p=0.005) and MDA-MB-231 (̂p=0.003) to form foci (FIGS. 3C, 3D); the ability of SUM159 (̂p<0.0001) and MDA-MB-231 (̂p<0.0001) to invade through Matrigel (FIGS. 3E, 3F); the ability of SUM159 (̂p<0.014) to laterally migrate in a wound healing assay (FIG. G); and the ability of SUM159 (̂p<0.02) to grow under anchorage independent conditions (FIG. 3H).

When injected into the mammary fat pad of female athymic nude mice, Merlin-expressing SUM159 cells showed notable (p<0.05) latency in the appearance of palpable tumors (FIG. 3I). The tumor size was represented as mean tumor diameter (̂p<0.0001 relative to vector controls; 4 mice were assessed per group). Tumors formed by vector control cells were evident beginning at 10 days post-injection, those formed by the mixed pool and clone 6 were palpable 19 days and 54 days after injection, respectively. The Merlin-transfectant A1 and A2 clones of MDA-MB-231 also demonstrated a significantly (p<0.05) reduced growth rate (FIG. 3J). The mixed pool of Merlin transfectants of both, SUM159 and MDA-MB-231 cells showed a modest, but significant reduction on tumor growth rate. This may be likely due to a mixed population of Merlin-expressing and nonexpressing cells. Cumulatively, restoration of Merlin expression in both breast cancer cell lines resulted in reduced malignant behavior.

### Example 5

### OPN Targets Merlin for Akt-Mediated Proteasomal Degradation

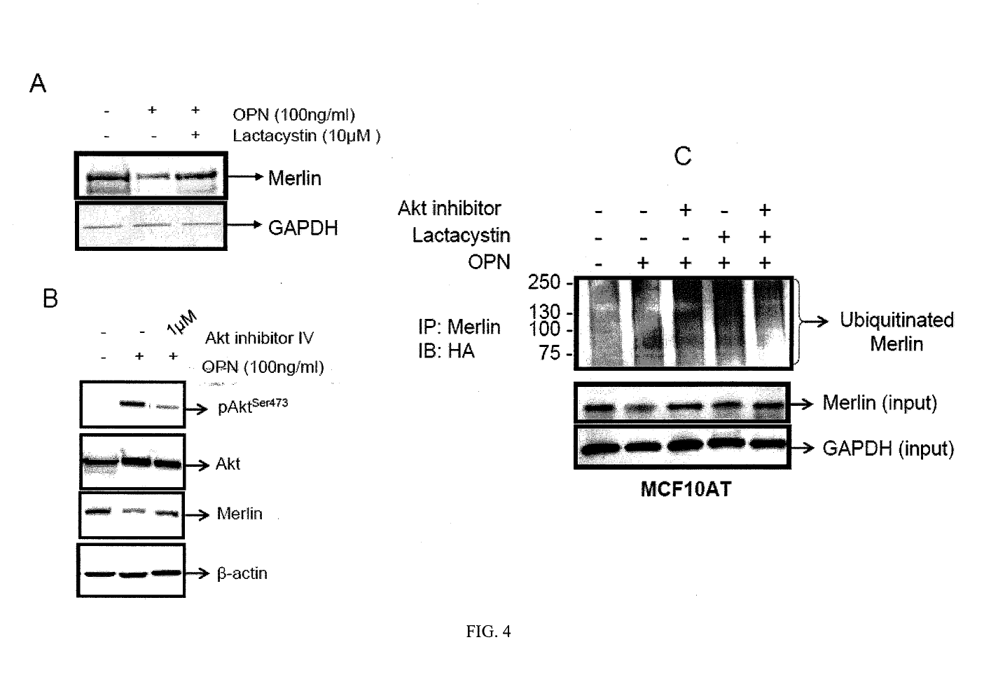

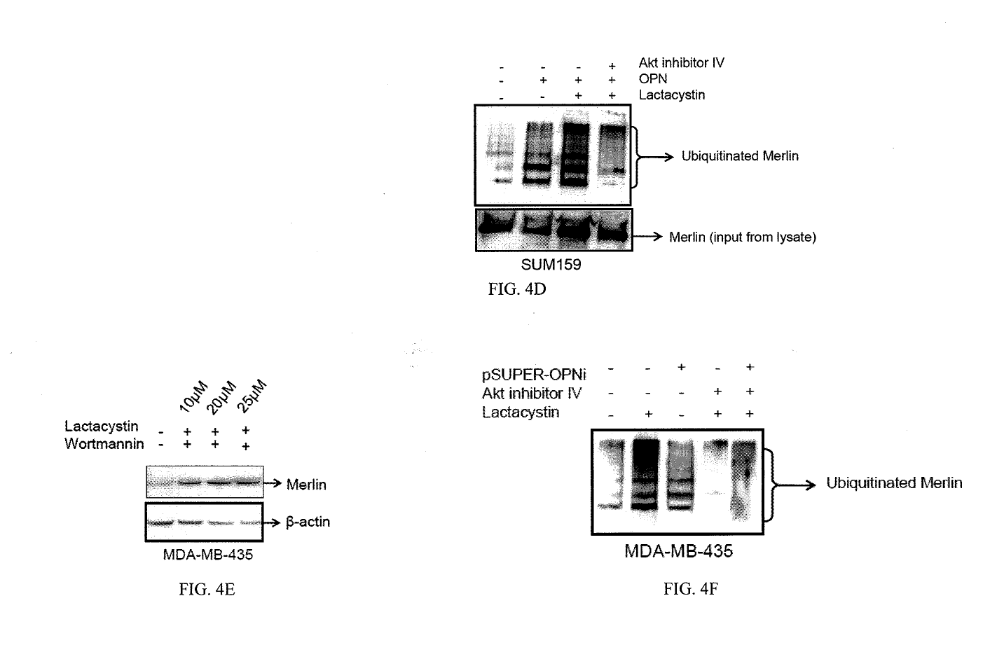

Akt signaling initiated downstream of OPN may regulate Merlin. To examine the effects of OPN on the post-translational regulation of Merlin, specifically the stability of Merlin protein, SUM159 breast cancer cells were transfected with Merlin cDNA and treated with recombinant OPN. OPN causes a decrease in the protein levels of Merlin (FIG. 4A). Treatment with the proteasome inhibitor, Lactacystin, rescued the levels of Merlin in OPN-treated cells, suggesting that OPN-initiated signaling targeted Merlin for proteasome-mediated degradation.

OPN interacts with a variety of cell surface receptors including CD44 and multiple integrins to activate signaling via the Akt pathway (31,38,39). To assess the role of Akt in OPN initiated degradation of Merlin, MCF10AT cells (which express Merlin but do not express detectable levels of OPN) were treated with recombinant OPN. Treatment with OPN activated Akt causing phosphorylation of Akt to phospho-Akt (Ser 417) concomitant with a decrease in the levels of Merlin suggesting that degradation of Merlin can be initiated by signaling downstream of OPN via Akt (FIG. 4B). The levels of total Akt remain unaltered. MCF10AT cells were also treated with Akt inhibitor IV in addition to OPN. While the levels of Akt phosphorylation predictably decreased after treatment, the levels of Merlin were restored by the inhibition of Akt phosphorylation even in the presence of OPN suggesting that inhibition of Akt activation blocks the effects on degradation of Merlin. Phosphorylation of Merlin via Akt targets it for degradation by the proteasome (15,40,41).

To examine if OPN can induce ubiquitination of endogenous Merlin leading to its proteasomal degradation, MCF10AT cells were transfected with a HA-ubiquitin expressing construct. MCF10AT cells were transfected with HA-ubiquitin and treated with OPN, Lactacystin and Akt inhibitor IV. Cell lysate (2 mg) harvested in NP40 buffer was immunoprecipitated overnight for endogenous Merlin. The immunoprecipitate was immunoblotted with anti-HA antibody. Merlin and GAPDH levels from the cell lysates were inputs for the experiment. In the presence of OPN, Merlin undergoes some ubiquitination that is evident as a smear (FIG. 4C). This smear persisted in the presence of Lactacystin, suggesting that Merlin was likely ubiquitinated in the cells in the presence of OPN.

To assess the role of activated Akt induced by OPN, cells were co-treated with an Akt inhibitor. SUM159 cells were transfected with HA-ubiquitin and Merlin and treated with OPN (100 ng/ml), Lactacystin (10 μM) and Akt inhibitor IV. Cell lysate (1 mg) harvested in NP40 buffer was immunoprecipitated for Merlin. The immunoprecipitate was immunoblotted with anti-HA antibody. Ubiquitination of Merlin was abolished in the presence of Akt inhibitor, suggesting that OPN-induced Akt phosphorylation caused degradation of endogenous Merlin via the ubiquitin-proteasome pathway. Similar results were observed in SUM159 cells constitutively expressing Merlin. Merlin ubiquitination was enhanced when co-treated with OPN and was abolished in the presence of Akt inhibitor re-affirming the role of Akt downstream of OPN in modulating the stability of Merlin (FIG. 4D).

The converse was seen when MDAMB-435 cells were treated with the proteasome inhibitor, Lactacystin (10-25 μM) and the PI-3-kinase inhibitor, wortmannin (100 nM). The MDA-MB-435 cells do not express detectable levels of Merlin, but express abundant OPN. Combined treatment with Lactacystin and wortmannin restored Merlin expression in the cells, suggesting that the PI-3-kinase/Akt pathway, in conjunction with the activities of the proteasome, regulates the protein levels of Merlin in the cells (FIG. 4E). Silencing the expression of OPN reduced the overall levels of ubiquitinated Merlin; in combination with Akt inhibitor and Lactacystin, abrogating OPN expression caused a notable decrease in the ubiquitinated Merlin (FIG. 4F).

### Example 6

### OPN Initiated Signaling Causes Phosphorylation of Merlin at Serine 315

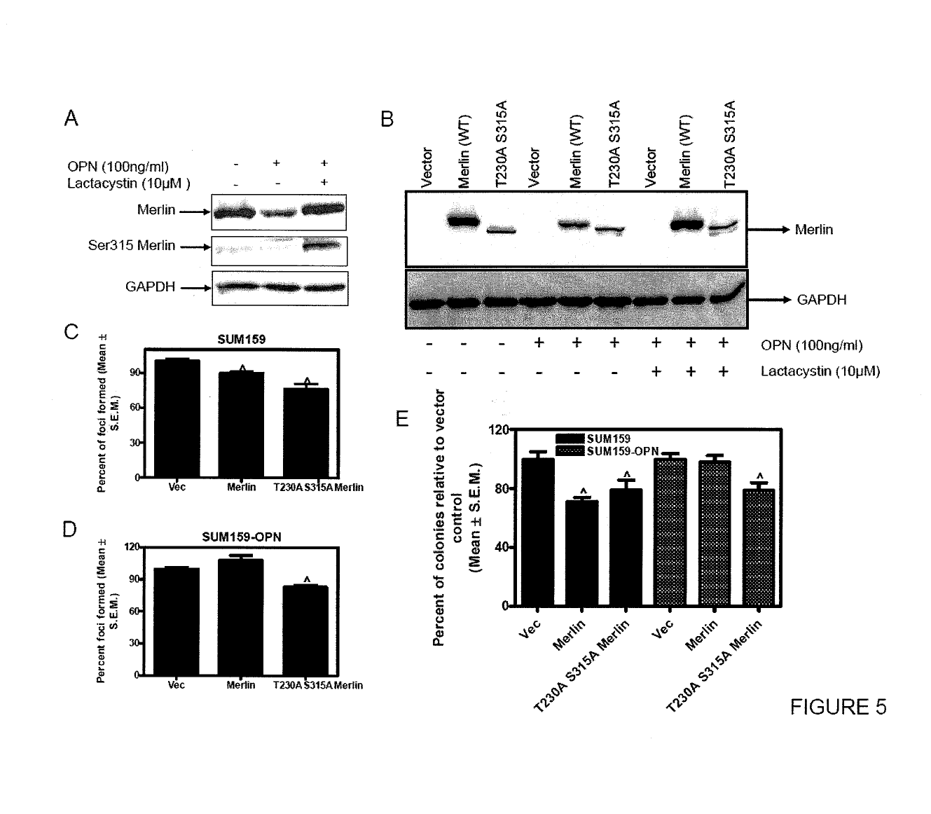

Lysate from SUM159 cells transfected with Merlin and treated with OPN was probed for total Merlin and phosphorylated Merlin (Serine 315). GAPDH was used as a loading control. The decrease in Merlin expression in presence of OPN was caused by the phosphorylation of Merlin at the Ser315 position (FIG. 5A). Phosphorylation of Merlin at this residue has been reported to target it for proteasome-mediated degradation (15,40). In particular, phosphorylation of Serine 315 and Threonine 230 makes Merlin refractory to OPN. SUM159 cells were transfected with Merlin (WT) or the T230A/S315A Merlin mutant and treated with OPN and Lactacystin. Cell lysates were probed for total Merlin. GAPDH was used as a loading control. Mutant Merlin (T230A S315A) is not degraded in response to OPN, whereas wild-type Merlin is degraded by OPN. Thus, phosphorylated Merlin was detectable upon inhibition of proteasomal degradation with Lactacystin in presence of OPN. It was further determined that while OPN is able to induce degradation of Merlin, the Merlin mutant T230A S315A (that cannot be phosphorylated by Akt) is resistant to the effects of OPN (FIG. 5B). Thus, cumulatively, these results suggest that OPN activates Akt-mediated signaling that causes phosphorylation of Merlin at Ser315. This event targets Merlin for ubiquitin-mediated degradation in breast cancer cells.

### Example 7

### Degradation-Resistant Merlin Functionally Restricts Malignant Behavior

The ability of the Merlin mutant T230A S315A for its ability to impact the properties of breast cancer cells in the perspective of OPN signaling was assessed. Wild-type Merlin and T230A S315A Merlin mutant can significantly (̂p<0.05) reduce foci formation ability of SUM159 cells. Plasmids corresponding to empty-vector, wild-type Merlin and Merlin mutant were transfected into SUM159 cells. Cells were detached and re-seeded in media containing selection antibiotics. Foci were counted after 10-14 days.

The wild-type Merlin and the T230A S315A Merlin mutant significantly (p<0.05) reduced the numbers of foci formed by the SUM159 cells (FIG. 5C). In order to test the effectiveness of T230A S315A Merlin mutant under conditions of elevated OPN expression, the ability of Merlin to impact the foci formation capability of SUM159-OPN (stably expressing OPN) cells was tested. While wild-type Merlin cannot impact the foci formation capability of the SUM159-OPN cells, the T230A S315A Merlin mutant brings about a significant (p<0.05) reduction in the numbers of foci formed (FIG. 5D). Similar results were obtained in the assessment of anchorage independent growth in a soft-agar colonization assay (FIG. 5E), in which wild-type Merlin and T230A S315A Merlin mutant can significantly reduce colony formation in soft agar by SUM159 cells (̂p<0.05). Only the degradation-resistant T230A S315A Merlin mutant reduced the ability to grow under anchorage-independent condition in soft agar in presence of elevated OPN signaling in SUM159 cells (̂p<0.05, relative to vector control). This suggests that the degradation resistant T230A S315A Merlin mutant retains its ability to effectively blunt malignant attributes in presence of OPN.

### Example 8

### OPN Enhances Tissue Identification and Discriminatory Power of Merlin

In order to assess the discriminatory power of Merlin and OPN, a logistic regression model was applied to a binary variable of normal and tumor tissue to data described herein. The Chi-square test for appropriateness of model (p=0.0448; ROC (Receiver Operating Characteristic) curve area=0.7220) indicates that Merlin has a discriminatory power for distinguishing between normal and tumor tissues (FIG. 6A). The logistic regression also showed that OPN by itself is not a good discriminator between normal and tumor tissues (p=0.2878; ROC area=0.6040) (FIG. 6B). Further, multiple logistic regression showed that OPN does not increase the discriminatory power of Merlin (p=0.162; ROC area=0.723) (FIG. 6C). Towards the possibility of developing a model that uses the unique inverse relationship between OPN and Merlin, a logistic regression model was applied to a selected cohort of the data, scoring only the positive staining events from normal tissues for Merlin and the positive staining events from tumor tissue for OPN. As seen in FIG. 6D, it is apparent that the logistic model for Merlin alone, using this data set is very good at discriminating between normal and breast tumor tissues (p<0.0001; R2=0.43; ROC area=0.93). Furthermore, given the Merlin intensity, OPN expression enhances tissue identification with increased discriminative power of the model (n=46; p<0.0001; R2=0.81; ROC area=0.9917) (FIG. 6E). A model developed from this training set was applied to the selected data and it was found that out of the 46 samples queried, only 2 samples were misclassified (FIG. 6F) resulting in 96% probability of correct classification.

### Example 9

### OPN mRNA and Merlin mRNA Expression

The relative levels of OPN and Merlin were measured in Hyperplastic Enlarged Lobular Units (HELU) compared to the Normal Terminal Duct Lobular Units (NTDLU) (8 samples, each), and in cases of Infiltrating Ductal Carcinoma (IDC) and Infiltrating Lobular Carcinoma (ILC) (10 samples total) compared to Lobular control (LC) and Ductal control (DC) cells (21 samples total). The NCBI GEO databases were used to derive information on the transcript levels of Merlin and OPN. The specific databases profiled were: GDS2739/g5730865_{—}3p_a_at/NF2/Homo sapiens; GDS2739/g189150_{—}3p_a_at/SPP1/Homo sapiens; GDS2635/217150_s_at/NF2/Homo sapiens; and GDS2635/209875_s_at/SPP1/Homo sapiens.

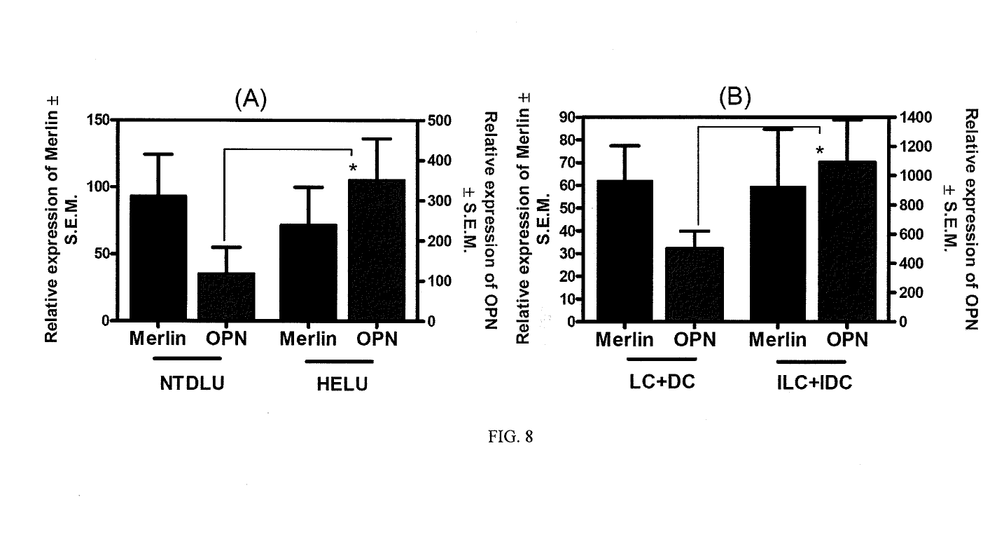

The relative levels of OPN significantly increased (p=0.0289) in HELU compared to the NTDLU. In contrast, the relative levels of Merlin were comparable in both NTDLU and HELU (FIG. 8, left panel). The expression of Merlin remained unaltered in cases of IDC and ILC compared to LC and DC cells (FIG. 8, right panel). In contrast, OPN levels significantly increased (p=0.0329) in ILC and IDC relative to control cases. Thus, an increase in OPN mRNA expression was not accompanied by a corresponding significant change in the mRNA levels of Merlin.

### Example 10

### Merlin Suppresses the Activity of the OPN Promoter and β-Catenin Promoter

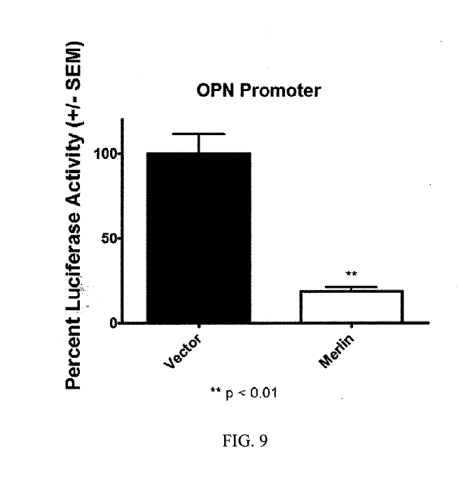

SUM159 cells were co-transfected with luciferase reporter constructs containing the OPN promoter and expression constructs containing Merlin, or control expression constructs. 33-40 hrs post-transfection cells were lysed overnight and assessed for luciferase activity. Data was normalized to total protein concentration. Expression of Merlin in SUM159 cells suppressed activity of the OPN promoter (FIG. 9).

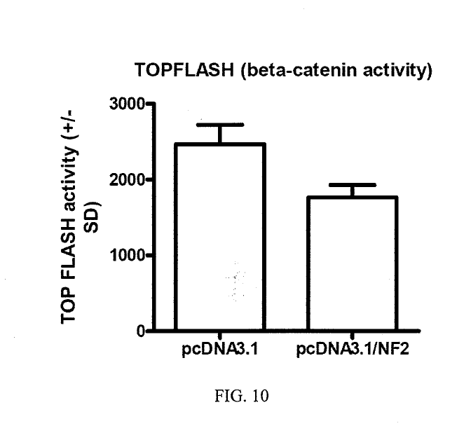

SUM159 cells were co-transfected with a TOPFLASH reporter constructs containing the β-catenin promoter and expression constructs containing Merlin (pcDNA3.1/NF2), or control expression constructs (pcDNA3.1). 33-40 hrs post-transfection cells were lysed overnight and assessed for TOPFLASH activity. Data was normalized to total protein concentration. Expression of Merlin in SUM159 cells suppressed activity of the β-catenin promoter (FIG. 10). The foregoing assays were also done with similar results in SUM159 cells stably transfected with Merlin.

### Example 11

### Merlin Causes Relocalization of β-Catenin from Nucleus to Cytosol

SUM159 cells expressing Merlin or control cells were washed with chilled PBS three times and fixed in 4% paraformaldehyde for 20 mins at ambient temperature and washed thrice again in chilled PBS. Cells were permeabilized in PBS containing 0.1% Triton X100 for 5-10 mins followed by three washed in chilled PBS. Cells were blocked in 1% BSA in 0.1% PBS-Triton X100 for 30 mins followed by incubation with primary antibodies for β-catenin and Merlin at 4° C. The following day, cells were washed thrice in PBS-Triton X100 followed by incubation with fluorophore-tagged secondary antibody at 37° for 90 mins in the dark. Lastly, cells were washed again and mounted in DAPI (vectastain) and imaged on Nikon TE2000 (40×; 1.5×). In control cells, β-catenin was distributed in the nucleus (FIG. 11). In cells overexpressing Merlin, β-catenin was distributed throughout the cell (FIG. 11).

### Example 12

### Merlin Knockdown Causes β-Catenin Relocalization from Membrane to Cytosol

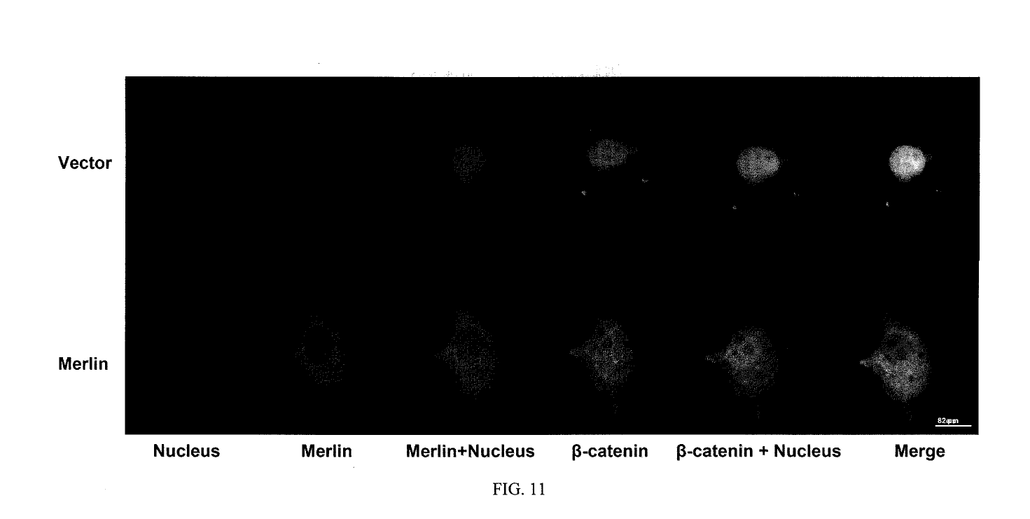

MCF7 cells with transfected with a Merlin knockdown construct or a control knockdown construct were prepared. The distribution of β-catenin in the Merlin knockdown cells and control cells was visualized as described above. In Merlin knockdown cells, β-catenin remained distributed in the cell nucleus (FIG. 12).

### Example 13

### Restoration of Merlin does not Significantly Affect β-Catenin mRNA Levels

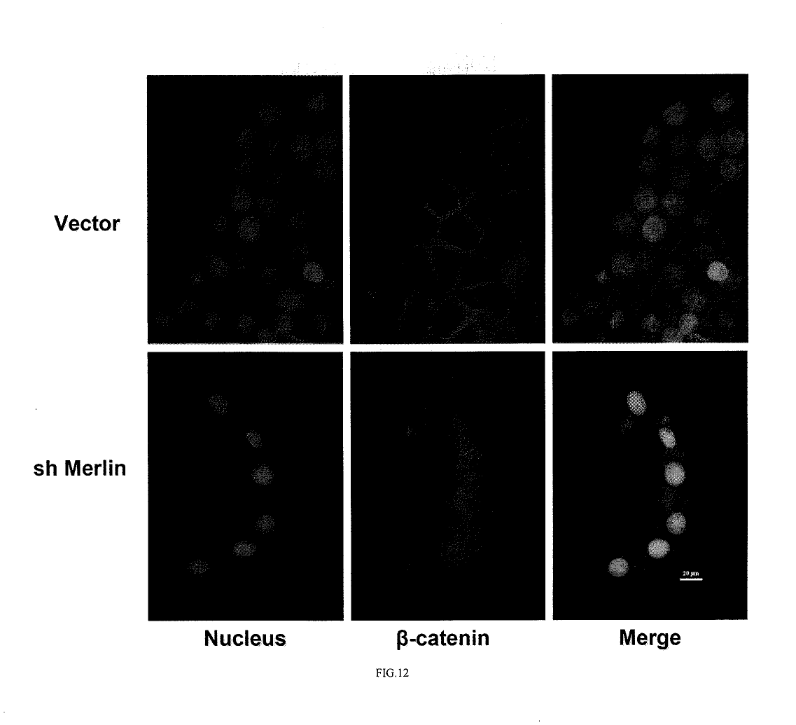

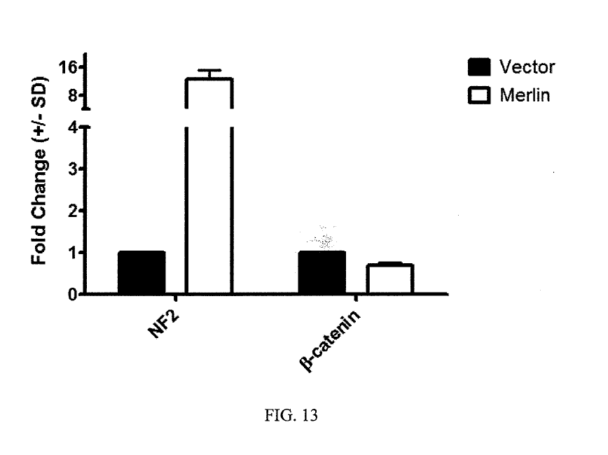

Merlin and β-catenin mRNA levels were measured in cells transfected with either a Merlin expression construct or a control expression construct. Real time quantitative RT-PCR: 1 μg of total RNA was used to synthesize cDNA (High Capacity Reverse Transcription kit from Applied Biosystems, Foster City, Calif.). PCR was performed using 40 ng of cDNA with β-catenin TaqMan primer probe sets in TaqMan Universal PCR Master Mix (Applied Biosystems) using a BioRad iQ5Real-Time Detection system (Bio-Rad, Hercules, Calif.) The transcript levels were normalized to GAPDH levels. Restoration of Merlin did not significantly affect β-catenin mRNA levels (FIG. 13).

### Example 14

### Merlin Interacts with β-Catenin

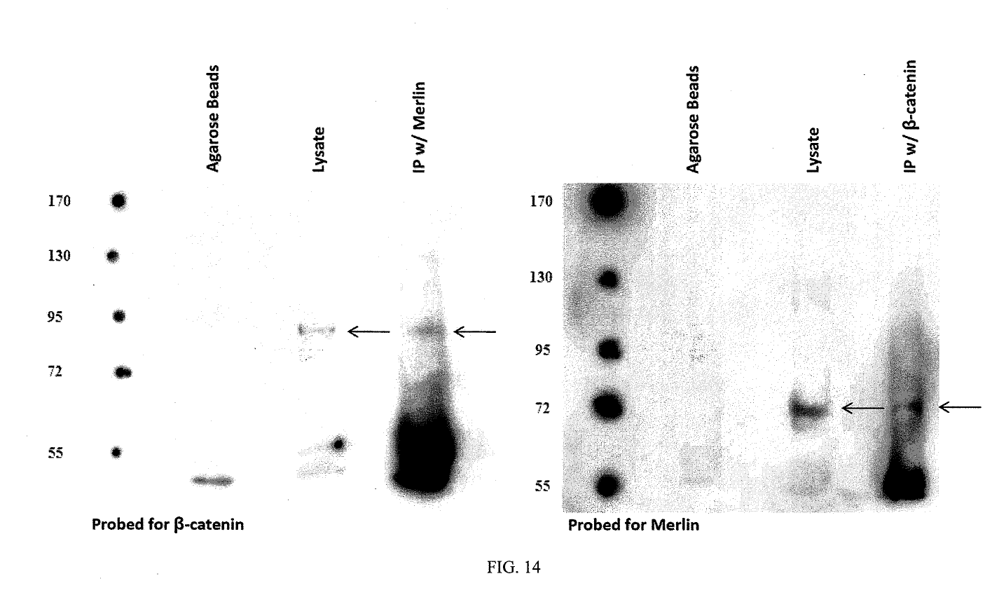

The interaction of Merlin and β-catenin in SUM159 cells was investigated by immunoprecipitation. The lysate of SUM159-Merlin transfectant cells was immunoprecipitated for Merlin or β-catenin and probed by immunoblotting, following SDS-PAGE for β-catenin or Merlin, respectively. Immunoprecipitation with Merlin and detection with β-catenin (FIG. 14, left panel; arrow indicates (β-catenin), or immunoprecipitation with β-catenin and detection with Merlin resulted in detectable species both resulted in detectable species (FIG. 14, right panel; arrow indicates Merlin). Thus, Merlin interacts with β-catenin as shown by immunoprecipitation.

### Example 15

### Enrichment of Spheroid-Forming Cells (SFC)

The growth of cancer cells as multicellular spheroids has frequently been reported to mimic the in vivo tumor architecture and physiology and has been utilized to study antitumor drugs. In order to determine the distinctive characteristics of the spheroid-derived cells compared to the corresponding monolayer-derived cells, multicellular spheroid-forming subpopulations of cells were enriched from three human breast cancer cell lines, namely, MCF7, MCF10AT and MCF10DCIS.com (Shevde L. A., et al., (2009) J. Cell. Mol. Med. 14:1693-1706, incorporated herein by reference in its entirety). MCF10AT cells provide a model of proliferative, pre-neoplastic breast (Dawson P. J. et al., (1996) Am J. Pathol 148:313-319, incorporated herein by reference in its entirety); MCF7 cells provide a model of an adenocarcinoma of the breast; and MCF10DCIS.com cells provide a model of lesions of Human Comedo Ductal Carcinoma.

Spheroid-forming cell (SFC) populations were derived from MCF10AT, MCF7 and MCFCF10DCIS.com cells by culturing each cell line under conditions of compromised adherence in low attachment tissue culture plates (Corning, Corning, N.Y.) in DMEM-F12 (Invitrogen) supplemented with 0.4% bovine serum albumin (BSA; Sigma), 25 ng/ml EGF (Sigma) and 10 ng/ml basic fibroblast growth factor (bFGF; Sigma). The effect of serum-starvation was studied by culturing the spheroids for 16 hrs in serum-free, phenol red-free medium followed by growth in the ambient medium for 48-72 hrs. Photographs were acquired at 10× magnification musing a Zeiss Axiocam 200M microscope (Carl Zeiss Microimaging, Gottingen, Germany). The spheroid forming cells enriched from MCF10DCIS.com (DCIS-SFC) were implanted into the mammary fat pad of female athymic nude mice.

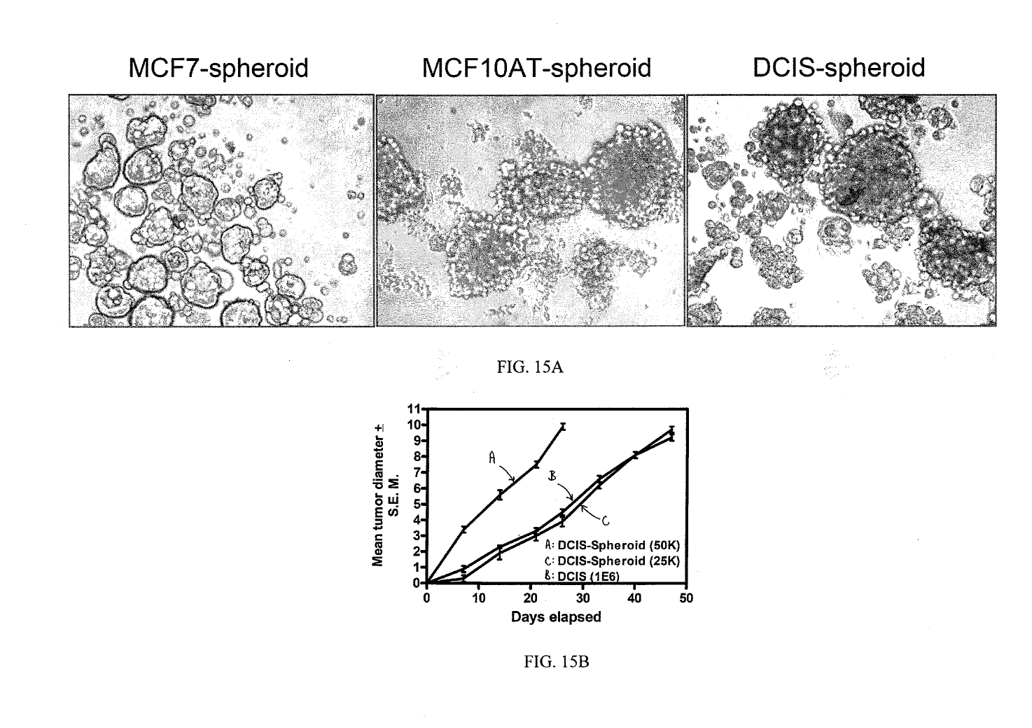

The enriched sub-populations of spheroid cells were highly tumorigenic. The SFCs derived from the three parent cell lines displayed differences in their morphology. The spheroids enriched from MCF7 cells appeared to comprise of cells packed more tightly in a compact structure than those from MCF10AT and MCF10DCIS.com (FIG. 15A). As few as 50,000 cells are able to form a rapidly growing tumour compared to the adherent MCF10DCIS.com cells (DCIS; 1 million cells injected) (FIG. 15B). The spheroid-forming cells derived from MCF7 cells (MCF7-SFC) display enhanced tumorigenic potential compared to the monolayer-derived adherent cells. As few as 1×10⁵ cells MCF7-SFC cells were able to form a tumor at the same rate (P=0.50) as that of the adherent MCF7 cells. Overall, the spheroid-enriched cells displayed enhanced tumorigenicity compared to the adherent monolayer-derived cells.

### Example 16

### Identification of Deregulated MicroRNAs in Spheroid-Forming Cells

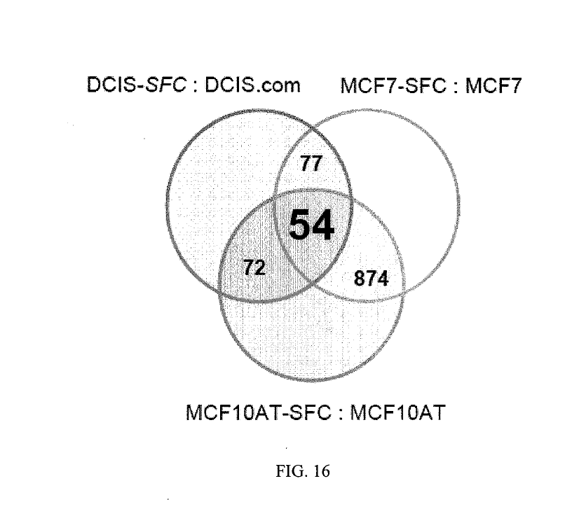

MicroRNAs (miRNAs) are short RNA molecules that include post-transcriptional regulators capable of binding to complementary sequences on target mRNAs. Aberrant expression of miRNAs has been implicated in several disease states. The miRNAs differentially regulated in the SFC cell lines, DCIS-SFC, MCF7-SFC, and MCF10AT-SFC, relative to their respective parent cell lines, namely, DCIS.com, MCF7, and MCF10-AT cell lines were identified. RNA samples were labeled using the miRCURY™ Hy3™/Hy5™ labeling kit and hybridized on the miRCURY™ LNA Array (v. 8.1). The number of differentially regulated miRNAs common between each SFC cell lines is shown in FIG. 16.

Fifty-four differentially regulated miRNAs were identified to be common to each SFC cell line. Of the fifty-four commonly de-regulated miRNAs, 43 miRNAs were upregulated in the SFC cell lines, and 11 miRNAs were downregulated in the SFC cell lines. Thus, it is evident that there exist common regulatory pathways that likely determine the lethal behavior of the SFCs. Table 4 lists the fold-change in levels of mature miRNAs in the spheroid forming cells, MCF10-DCIS-SFC, MCF7-SFC, and MCF10AT-SFC, relative to the levels in the parent monolayer derived adherent cell lines, MCF10-DCIS, MCF7, and MCF10AT, respectively. Some of the mature miRNAs listed in Table 4 include the 5-p and 3-p mature miRNAs of a precursor miRNA, some of the mature miRNAs listed in Table 4 include family members or related members of a cluster of miRNAs.

TABLE 4

| | Fold change in expression | | | | |
|---|---|---|---|---|---|
| | level in cell line | | | | |
| | MCF7 | MCF10AT | DCIS.com | | SEQ ID |
| miRNA | cells | cells | cells | Mature miRNA sequence | NO. |
| hsa-let-7c | 100 | 1.43 | 100 | UGAGGUAGUAGGUUGUAUGGUU | SEQ ID |
| ↑ | | | | | NO: 01 |
| hsa-miR- | 1.16 | 1.1 | 3.94 | [hsa-miR-296-5p] | SEQ ID |
| 296 ↑ | | | | AGGGCCCCCCCUCAAUCCUGU | NO: 02 |
| | | | | [hsa-miR-296-3p] | SEQ ID |
| | | | | GAGGGUUGGGUGGAGGCUCUCC | NO: 03 |
| hsa-let-7d | 1.52 | 1.65 | 2.78 | AGAGGUAGUAGGUUGCAUAGUU | SEQ ID |
| ↑ | | | | | NO: 04 |
| hsa-miR- | 1.99 | 1.23 | 2.63 | UGUAAACAUCCUACACUCUCAGC | SEQ ID |
| 30c ↑ | | | | | NO: 05 |
| hsa-miR- | 1.84 | 1.14 | 2.45 | UAAAGUGCUUAUAGUGCAGGUAG | SEQ ID |
| 20a ↑ | | | | | NO: 06 |
| hsa-miR- | 1.21 | 1.17 | 2.18 | UGAAACAUACACGGGAAACCUC | SEQ ID |
| 494 ↑ | | | | | NO: 07 |
| hsa-miR- | 1.15 | 1.22 | 2.16 | [hsa-miR-320a] | SEQ ID |
| 320 ↑ | | | | AAAAGCUGGGUUGAGAGGGCGA | NO: 08 |
| | | | | [hsa-miR-320b] | SEQ ID |
| | | | | AAAAGCUGGGUUGAGAGGGCAA | NO: 09 |
| | | | | [hsa-miR-320c] | SEQ ID |
| | | | | AAAAGCUGGGUUGAGAGGGU | NO: 10 |
| | | | | [hsa-miR-320d] | SEQ ID |
| | | | | AAAAGCUGGGUUGAGAGGA | NO: 11 |
| | | | | [hsa-miR-320e] | SEQ ID |
| | | | | AAAGCUGGGUUGAGAAGG | NO: 12 |
| hsa-miR- | 1.3 | 1.25 | 2.12 | UUUCAAGCCAGGGGGCGUUUUUC | SEQ ID |
| 498 ↑ | | | | | NO: 13 |
| hsa-miR- | 1.09 | 1.03 | 2.07 | GAAGUUGUUCGUGGUGGAUUCG | SEQ ID |
| 382 ↑ | | | | | NO: 14 |
| hsa-miR- | 1.05 | 1.20 | 2.05 | UGGAGAGAAAGGCAGUUCCUGA | SEQ ID |
| 185 ↑ | | | | | NO: 15 |
| hsa-miR- | 2.24 | 1.45 | 2.00 | CACCCGUAGAACCGACCUUGCG | SEQ ID |
| 99b ↑ | | | | | NO: 16 |
| hsa-let-7a | 2.21 | 2.45 | 1.86 | UGAGGUAGUAGGUUGUAUAGUU | SEQ ID |
| ↑ | | | | | NO: 17 |
| hsa-miR- | 1.04 | 1.05 | 1.82 | CUGCAAAGGGAAGCCCUUUC | SEQ ID |
| 527 ↑ | | | | | NO: 18 |
| hsa-miR- | 1.42 | 1.64 | 1.79 | UAGCAGCACAGAAAUAUUGGC | SEQ ID |
| 195 ↑ | | | | | NO: 19 |
| hsa-miR- | 1.16 | 1.01 | 1.78 | UACUCAGGAGAGUGGCAAUCAC | SEQ ID |
| 510 ↑ | | | | | NO: 20 |
| hsa-miR- | 1.13 | 1.04 | 1.77 | CAGUGCAAUGAUGAAAGGGCAU | SEQ ID |
| 130b ↑ | | | | | NO: 21 |
| hsa-miR- | 1.38 | 1.08 | 1.75 | [hsa-miR-361-5p] | SEQ ID |
| 361 ↑ | | | | UUAUCAGAAUCUCCAGGGGUAC | NO: 22 |
| | | | | [hsa-miR-361-3p] | SEQ ID |
| | | | | UCCCCCAGGUGUGAUUCUGAUUU | NO: 23 |
| hsa-miR- | 1.03 | 1.09 | 1.68 | UGGACGGAGAACUGAUAAGGGU | SEQ ID |
| 184 ↑ | | | | | NO: 24 |
| hsa-miR- | 1.04 | 1.04 | 1.67 | GGUCCAGAGGGGAGAUAGGUUC | SEQ ID |
| 198 ↑ | | | | | NO: 25 |
| hsa-miR- | 1.82 | 1.34 | 1.61 | AAUCGUACAGGGUCAUCCACUU | SEQ ID |
| 487b ↑ | | | | | NO: 26 |
| hsa-let-7b | 1.37 | 1.47 | 1.60 | UGAGGUAGUAGGUUGUGUGGUU | SEQ ID |
| ↑ | | | | | NO: 27 |
| hsa-miR- | 1.06 | 1.18 | 1.57 | ACAGCAGGCACAGACAGGCAGU | SEQ ID |
| 214 ↑ | | | | | NO: 28 |
| hsa-miR- | 1.27 | 1.06 | 1.56 | UGUCUGCCCGCAUGCCUGCCUCU | SEQ ID |
| 346 ↑ | | | | | NO: 29 |
| hsa-let-7e | 1.27 | 1.36 | 1.54 | UGAGGUAGGAGGUUGUAUAGUU | SEQ ID |
| ↑ | | | | | NO: 30 |
| hsa-miR- | 1.01 | 1.01 | 1.54 | UGAGGUAGGAGGUUGUAUAGUU | SEQ ID |
| 335 ↑ | | | | | NO: 31 |
| hsa-miR- | 1.49 | 1.53 | 1.53 | [hsa-miR-125a-5p] | SEQ ID |
| 125a ↑ | | | | UCCCUGAGACCCUUUAACCUGUGA | NO: 32 |
| | | | | [hsa-miR-125a-3p] | SEQ ID |
| | | | | ACAGGUGAGGUUCUUGGGAGCC | NO: 33 |
| hsa-miR- | 1.22 | 1.22 | 1.51 | CAAAGUGCCUCCCUUUAGAGUG | SEQ ID |
| 519d ↑ | | | | | NO: 34 |
| hsa-miR- | 1.19 | 1.10 | 1.51 | [hsa-miR-423-5p] | SEQ ID |
| 423 ↑ | | | | UGAGGGGCAGAGAGCGAGACUUU | NO: 35 |
| | | | | [hsa-miR-423-3p] | SEQ ID |
| | | | | AGCUCGGUCUGAGGCCCCUCAGU | NO: 36 |
| hsa-miR- | 1.10 | 1.24 | 1.49 | UAGCAGCGGGAACAGUUCUGCAG | SEQ ID |
| 503 ↑ | | | | | NO: 37 |
| hsa-miR- | 1.02 | 1.01 | 1.48 | CGCAUCCCCUAGGGCAUUGGUGU | SEQ ID |
| 324-5p ↑ | | | | | NO: 38 |
| hsa-miR- | 1.03 | 1.04 | 1.47 | [hsa-miR-518f*] | SEQ ID |
| 518P-hsa- | | | | CUCUAGAGGGAAGCACUUUCUC | NO: 39 |
| miR-526a | | | | [hsa-miR-518e*, hsa-miR-518d-5p] | SEQ ID |
| cluster ↑ | | | | CUCUAGAGGGAAGCACUUUCUG | NO: 40 |
| | | | | [hsa-miR-519a*; hsa-miR-519b-5p; | SEQ ID |
| | | | | hsa-miR-519c-5p] | NO: 41 |
| | | | | CUCUAGAGGGAAGCGCUUUCUG | |
| | | | | [hsa-miR-520c-5p] | SEQ ID |
| | | | | CUCUAGAGGGAAGCACUUUCUG | NO: 42 |
| | | | | [hsa-miR522*; hsa-miR-523*] | SEQ ID |
| | | | | CUCUAGAGGGAAGCGCUUUCUG | NO: 43 |
| | | | | [hsa-miR-526a] | SEQ ID |
| | | | | CUCUAGAGGGAAGCACUUUCUG | NO: 44 |
| hsa-miR- | 1.07 | 1.12 | 1.44 | CUCUUGAGGGAAGCACUUUCUGU | SEQ ID |
| 526b ↑ | | | | | NO: 45 |
| hsa-miR- | 1.21 | 1.10 | 1.42 | UGUGACUGGUUGACCAGAGGGG | SEQ ID |
| 134 ↑ | | | | | NO: 46 |
| hsa-miR- | 1.03 | 1.11 | 1.40 | AACUGUUUGCAGAGGAAACUGA | SEQ ID |
| 452 ↑ | | | | | NO: 47 |
| hsa-miR- | 1.21 | 1.17 | 1.40 | UGAAACAUACACGGGAAACCUC | SEQ ID |
| 494 ↑ | | | | | NO: 48 |
| hsa-miR- | 1.03 | 1.05 | 1.36 | AGAGGUAUAGGGCAUGGGAA | SEQ ID |
| 202 ↑ | | | | | NO: 49 |
| hsa-miR- | 1.04 | 1.14 | 1.35 | [hsa-miR-513-5p] | SEQ ID |
| 513 ↑ | | | | UUCACAGGGAGGUGUCAU | NO: 50 |
| | | | | [hsa-miR-513-3p] | SEQ ID |
| | | | | UAAAUUUCACCUUUCUGAGAAGG | NO: 51 |
| hsa-miR- | 1.37 | 1.19 | 1.35 | [hsa-miR-490-5p] | SEQ ID |
| 490 ↑ | | | | CCAUGGAUCUCCAGGUGGGU | NO: 52 |
| | | | | [hsa-miR-490-3p] | SEQ ID |
| | | | | CAACCUGGAGGACUCCAUGCUG | NO: 53 |
| hsa-miR- | 1.15 | 1.06 | 1.35 | [hsa-miR-208a] | SEQ ID |
| 208 ↑ | | | | AUAAGACGAGCAAAAAGCUUGU | NO: 54 |
| | | | | [hsa-miR-208b] | SEQ ID |
| | | | | AUAAGACGAACAAAAGGUUUGU | NO: 55 |
| hsa-miR- | 1.05 | 1.06 | 1.30 | [hsa-miR-525-5p] | SEQ ID |
| 525 ↑ | | | | CUCCAGAGGGAUGCACUUUCU | NO: 56 |
| | | | | [hsa-miR-525-3p] | SEQ ID |
| | | | | GAAGGCGCUUCCCUUUAGAGCG | NO: 57 |
| hsa-miR-24 | 1.76 | 1.08 | 1.26 | [hsa-miR-24-1] | SEQ ID |
| ↑ | | | | UGGCUCAGUUCAGCAGGAACAG | NO: 58 |
| | | | | [hsa-miR-24-2] | SEQ ID |
| | | | | UGGCUCAGUUCAGCAGGAACAG | NO: 59 |
| hsa-miR- | 2.03 | 1.27 | 1.25 | AUCACAUUGCCAGGGAUUACC | SEQ ID |
| 23b ↑ | | | | | NO: 60 |
| hsa-miR- | 1.63 | 1.19 | 1.15 | AGCUACAUCUGGCUACUGGGU | SEQ ID |
| 222 ↑ | | | | | NO: 61 |
| hsa-miR- | 0.89 | 0.89 | 0.99 | UGCUUCCUUUCAGAGGGU | SEQ ID |
| 516-3p ↓ | | | | | NO: 62 |
| hsa-miR- | 0.80 | 0.83 | 0.8 | AUCACACAAAGGCAACUUUUGU | SEQ ID |
| 377 ↓ | | | | | NO: 63 |
| hsa-miR- | 0.95 | 0.98 | 0.79 | [hsa-miR-199a-5p] | SEQ ID |
| 199a ↓ | | | | CCCAGUGUUCAGACUACCUGUUC | NO: 64 |
| | | | | [hsa-miR-199a-3p] | SEQ ID |
| | | | | ACAGUAGUCUGCACAUUGGUUA | NO: 65 |
| hsa-miR- | 0.80 | 0.8 | 0.59 | UUAAUGCUAAUCGUGAUAGGGGU | SEQ ID |
| 155 ↓ | | | | | NO: 66 |
| hsa-miR- | 0.85 | 0.83 | 0.001 | UGUCAGUUUGUCAAAUACCCCA | SEQ ID |
| 223 ↓ | | | | | NO: 67 |
| hsa-miR- | 0.86 | 0.92 | 0.001 | UGGUUUACCGUCCCACAUACAU | SEQ ID |
| 299-5p ↓ | | | | | NO: 68 |
| hsa-miR- | 0.80 | 0.83 | 0.001 | CCUAGUAGGUGUCCAGUAAGUGU | SEQ ID |
| 325 ↓ | | | | | NO: 69 |
| hsa-miR- | 0.93 | 0.97 | 0.001 | AAUUGCACUUUAGCAAUGGUGA | SEQ ID |
| 367 ↓ | | | | | NO: 70 |
| hsa-miR- | 0.76 | 0.74 | 0.001 | UGGUAGACUAUGGAACGUAGG | SEQ ID |
| 379 ↓ | | | | | NO: 71 |
| hsa-miR- | 0.78 | 0.72 | 0.001 | [hsa-miR-323b-5p] | SEQ ID |
| 453 ↓ | | | | AGGUUGUCCGUGGUGAGUUCGCA | NO: 72 |
| | | | | [hsa-miR-323-3p] | SEQ ID |
| | | | | CCCAAUACACGGUCGACCUCUU | NO: 73 |
| hsa-miR- | 0.78 | 0.79 | 0.001 | AAAAUGGUUCCCUUUAGAGUGU | SEQ ID |
| 522 ↓ | | | | | NO: 74 |

↑: upregulated miRNA
↓: downregulated miRNA

Upregulated miRNAs were identified that were predicted to target Merlin mRNA using the MicroCosm Targets tool (miRBase Targets Release Version v5, <http:micrrna.sanger.ac.uk/targets/>). A targeting score for each Merlin targeting miRNA was calculated using the miRanda algorithm that takes into account complementarity alignment in a double stranded antiparallel duplex. The overall score for a hit is the summation of the derived scores across the total miRNA vs UTR alignment. Greater scores indicate better complementarity with the Merlin messenger RNA. As Merlin is a tumor suppressor, the 12 miRNAs that target Merlin are putative onco-miRNAs. Table 5 lists the identity of the Merlin targeting miRNAs and its targeting score.

TABLE 5

| Upregulated miRNA | SEQ ID NO |
|---|---|
| hsa-miR-513 | SEQ ID NO: 50 |
| hsa-miR-361 | SEQ ID NO: 22; SEQ ID NO: 23 |
| hsa-let-7e | SEQ ID NO: 30 |
| hsa-miR-526b | SEQ ID NO: 45 |
| hsa-let-7b | SEQ ID NO: 27 |
| hsa-let-7a | SEQ ID NO: 17 |
| hsa-let-7c | SEQ ID NO: 01 |
| hsa-miR-519d | SEQ ID NO: 34 |
| hsa-miR-24 | SEQ ID NO: 58; SEQ ID NO: 59 |
| hsa-miR-526a | SEQ ID NO: 44 |
| hsa-miR-195 | SEQ ID NO: 19 |
| hsa-miR-510 | SEQ ID NO: 20 |
| hsa-miR-184 | SEQ ID NO: 24 |

### Example 17

### Merlin Expression in Spheroid-Forming Cells

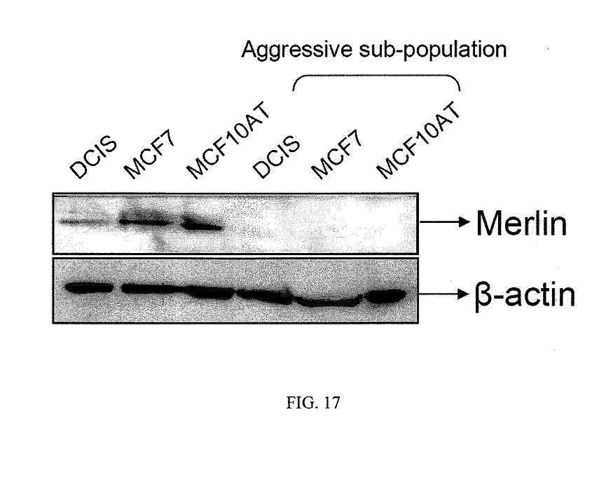

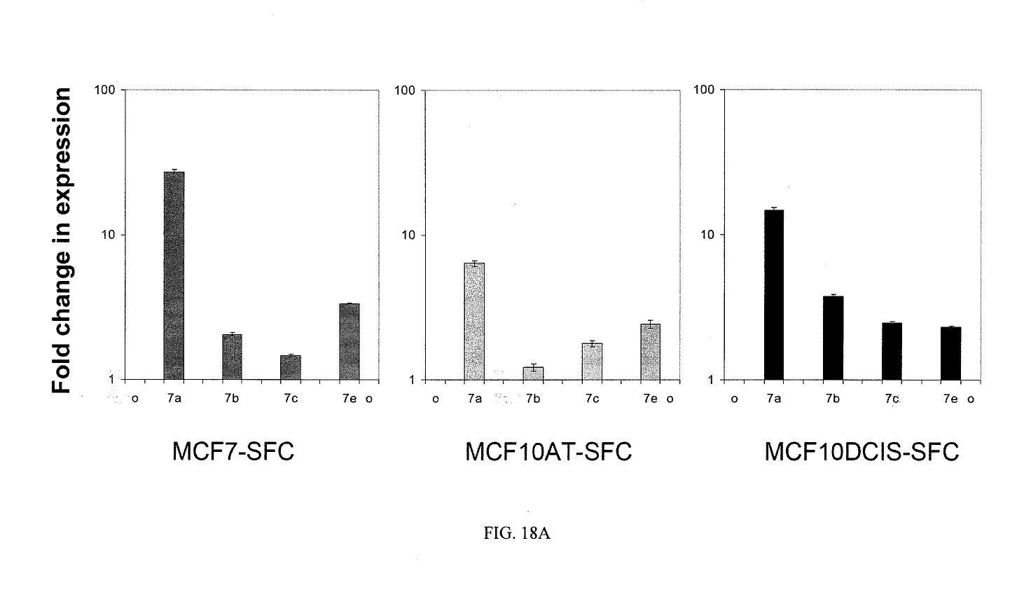

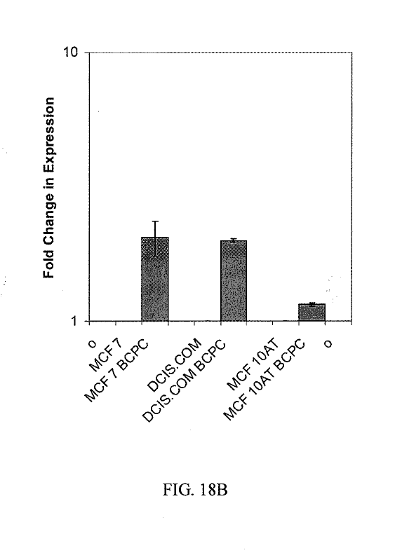

Protein expression of Merlin was measured in spheroid-forming cells enriched from DCIS.com, MCF7, and MCF10-AT cell lines. Cell lysates (30 μg) were resolved by SDS-PAGE and immunoblotted for Merlin. β-actin (Bio-Rad, Hercules, Calif., USA) was monitored as a loading control. Western analysis showed lack of Merlin expression in spheroid-forming cells enriched from DCIS.com, MCF7, and MCF10-AT cell lines, in contrast to the parent cell lines (FIG. 17). The relative level of expression of the miRNAs hsa-let-7a, hsa-let-7b, hsa-let-7c, hsa-let-7e, and mir-361 was measured using quantitative real-time PCR in the spheroid-forming cell lines DCIS-SFC, MCF7-SFC, and MCF10AT-SFC, relative to the level of each miRNA in the parent of each SFC-cell line, namely, DCIS.com, MCF7, and MCF10-AT, respectively (FIGS. 18A, 18B).

In sum, spheroid-forming cell populations (SFCs) from three established breast cancer cell lines were isolated. Each SFC cell line was ascertained to have an aggressive tumorigenic behavior and commonly deregulated miRNAs were identified. The target for 12 identified upregulated miRNAs includes Merlin; Merlin expression was decreased in each SFCs.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

### REFERENCES

1. Lau, Y. K., Murray, L. B., Houshmandi, S. S., Xu, Y., Gutmann, D. H., and Yu, Q. (2008) Cancer Res 68, 5733-5742
2. Rushing, E. J., Cooper, P. B., Quezado, M., Begnami, M., Crespo, A., Smirniotopoulos, J. G., Ecklund, J., Olsen, C., and Santi, M. (2007) J Neurooncol 85, 297-305
3. Muranen, T., Gronholm, M., Lampin, A., Lallemand, D., Zhao, F., Giovannini, M., and Carpen, O. (2007) Hum Mol Genet 16, 1742-1751
4. Begnami, M. D., Palau, M., Rushing, E. J., Santi, M., and Quezado, M. (2007) Hum Pathol 38, 1345-1350
5. Fouladi, M., Helton, K., Dalton, J., Gilger, E., Gajjar, A., Merchant, T., Kun, L., Newsham, I., Burger, P., and Fuller, C. (2003) Cancer 98, 2232-2244
6. James, M. F., Han, S., Polizzano, C., Plotkin, S. R., Manning, B. D., Stemmer-Rachamimov, A. O., Gusella, J. F., and Ramesh, V. (2009) Mol Cell Biol 29, 4250-4261
7. Sainio, M., Zhao, F., Heiska, L., Turunen, O., den Bakker, M., Zwarthoff, E., Lutchman, M., Rouleau, G. A., Jaaskelainen, J., Vaheri, A., and Carpen, O. (1997) J Cell Sci 110 (Pt 18), 2249-2260
8. Poulikakos, P. I., Xiao, G. H., Gallagher, R., Jablonski, S., Jhanwar, S. C., and Testa, J. R. (2006) Oncogene 25, 5960-5968
9. Kim, H., Lim, J. Y., Kim, Y. H., Park, S. H., Lee, K. H., Han, H., Jeun, S. S., Lee, J. H., and Rha, H. K. (2002) Mol Cells 14, 108-114
10. Morrison, H., Sherman, L. S., Legg, J., Banine, F., Isacke, C., Haipek, C. A., Gutmann, D. H., Ponta, H., and Herrlich, P. (2001) Genes Dev 15, 968-980
11. Morrison, H., Sperka, T., Manent, J., Giovannini, M., Ponta, H., and Herrlich, P. (2007) Cancer Res 67, 520-527
12. Xiao, G. H., Gallagher, R., Shetler, J., Skele, K., Altomare, D. A., Pestell, R. G., Jhanwar, S., and Testa, J. R. (2005) Mol Cell Biol 25, 2384-2394
13. Kissil, J. L., Johnson, K. C., Eckman, M. S., and Jacks, T. (2002) J Biol Chem 277, 10394-10399
14. Bai, Y., Liu, Y. J., Wang, H., Xu, Y., Stamenkovic, I., and Yu, Q. (2007) Oncogene 26, 836-850
15. Tang, X., Jong, S. W., Wang, X., Liu, Z., Bahr, S. M., Sun, S. Y., Brat, D., Gutmann, D. H., and Ye, K. (2007) Nat Cell Biol 9, 1199-1207
16. Nunes, F., Shen, Y., Niida, Y., Beauchamp, R., Stemmer-Rachamimov, A. O., Ramesh, V., Gusella, J., and MacCollin, M. (2005) Cancer Genet Cytogenet 162, 135-139
17. Wallace, A. J., Watson, C. J., Oward, E., Evans, D. G., and Elles, R. G. (2004) Genet Test 8, 368-380
18. Lasota, J., Wasag, B., Dansonka-Mieszkowska, A., Karcz, D., Millward, C. L., Rys, J., Stachura, J., Sobin, L. H., and Miettinen, M. (2003) Lab Invest 83, 1361-1371
19. Arakawa, H., Hayashi, N., Nagase, H., Ogawa, M., and Nakamura, Y. (1994) Hum Mol Genet 3, 565-568
20. Yaegashi, S., Sachse, R., Ohuchi, N., Mori, S., and Sekiya, T. (1995) Jpn J Cancer Res 86, 929-933
21. Craig, A. M., Nemir, M., Mukherjee, B. B., Chambers, A. F., and Denhardt, D. T. (1988) Biochem Biophys Res Commun 157, 166-173
22. Shevde, L. A., Das, S., Clark, D. W., and Samant, R. S. (2009) Curr Mol Med 9
23. Shevde, L. A., Samant, R. S., Paik, J. C., Metge, B. J., Chambers, A. F., Casey, G., Frost, A. R., and Welch, D. R. (2006) Clin Exp Metastasis 23, 123-133
24. Kim, Y. W., Park, Y. K., Lee, J., Ko, S. W., and Yang, M. H. (1998) J Korean Med Sci 13, 652-657
25. Tuck, A. B., and Chambers, A. F. (2001) J Mammary Gland Biol Neoplasia 6, 419-429
26. Tuck, A. B., O'Malley, F. P., Singhal, H., Harris, J. F., Tonkin, K. S., Kerkyliet, N., Saad, Z., Doig, G. S., and Chambers, A. F. (1998) Int J Cancer 79, 502-508
27. Rudland, P. S., Platt-Higgins, A., El-Tanani, M., De Silva Rudland, S., Barraclough, R., Winstanley, J. H., Howitt, R., and West, C. R. (2002) Cancer Res 62, 3417-3427
28. Furger, K. A., Menon, R. K., Tuck, A. B., Bramwell, V. H., and Chambers, A. F. (2001) Curr Mol Med 1, 621-632
29. Singhal, H., Bautista, D. S., Tonkin, K. S., O'Malley, F. P., Tuck, A. B., Chambers, A. F., and Harris, J. F. (1997) Clin Cancer Res 3, 605-611
30. Rodrigues, L. R., Teixeira, J. A., Schmitt, F. L., Paulsson, M., and Lindmark-Mansson, H. (2007) Cancer Epidemiol Biomarkers Prev 16, 1087-1097
31. Das, R., Mahabeleshwar, G. H., and Kundu, G. C. (2003) J Biol Chem 278, 28593-28606
32. Philip, S., Bulbule, A., and Kundu, G. C. (2004) Glycoconj J 21, 429-441
33. Rangaswami, H., Bulbule, A., and Kundu, G. C. (2006) Glycoconj J 23, 221-232
34. Metge, B. J., Frost, A. R., King, J. A., Dyess, D. L., Welch, D. R., Samant, R. S., and Shevde, L. A. (2008) Clin Exp Metastasis 25, 753-763
35. Lacroix, M. (2009) Cancer Chemother Pharmacol 63, 567
36. Rae, J. M., Creighton, C. J., Meck, J. M., Haddad, B. R., and Johnson, M. D. (2007) Breast Cancer Res Treat 104, 13-19
37. Chambers, A. F. (2009) Cancer Res 69, 5292-5293
38. Robertson, B. W., and Chellaiah, M. A. Exp Cell Res 316, 1-11
39. Zhang, G., He, B., and Weber, G. F. (2003) Mol Cell Biol 23, 6507-6519
40. Okada, M., Wang, Y., Jong, S. W., Tang, X., Neri, L. M., and Ye, K. (2009) Cancer Res 69, 4043-4051
41. Ye, K. (2007) Cell Adh Migr 1, 196-198
42. Dai, J. L., Wang, L., Sahin, A. A., Broemeling, L. D., Schutte, M., and Pan, Y. (2004) Oncogene 23, 8681-8687
43. Yang, Y., Kitagaki, J., Wang, H., Hou, D. X., and Perantoni, A. O. (2009) Cancer Sci 100, 24-28
44. Trotman, L. C., Wang, X., Alimonti, A., Chen, Z., Teruya-Feldstein, J., Yang, H., Pavletich, N. P., Carver, B. S., Cordon-Cardo, C., Erdjument-Bromage, H., Tempst, P., Chi, S. G., Kim, H. J., Misteli, T., Jiang, X., and Pandolfi, P. P. (2007) Cell 128, 141-156
45. Gao, C., Guo, H., Mi, Z., Grusby, M. J., and Kuo, P. C. (2007) J Immunol 178, 1870-1881

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-125. (canceled)

126. A method for evaluating the presence, absence or metastatic potential of a breast cancer in a subject comprising measuring the expression level of Merlin protein in a sample obtained from the subject.

127. The method of claim 126, further comprising comparing the expression level of Merlin in the sample to the expression level of Merlin protein in normal tissue, or cancerous tissue with a known metastatic potential.

128. The method of claim 127, wherein a decrease in the level of expression of Merlin is indicative of the presence or metastatic potential of the breast cancer.

129. The method of claim 126, further comprising measuring the expression level of a nucleic acid encoding OPN or the expression level of OPN protein in the sample.

130. The method of claim 129, wherein an increase in the expression level of a nucleic acid encoding OPN or expression level of OPN protein relative to a pre-determined expression level of a nucleic acid encoding OPN or expression level of OPN protein is indicative of the presence or metastatic potential of the breast cancer.

131. The method of claim 126, wherein the breast cancer comprises an infiltrating ductal carcinoma (IDC) or a distant metastasis.

132. A method for evaluating the presence, absence or metastatic potential of a breast cancer in a subject comprising measuring the expression level of a phosphorylated Merlin protein in a sample obtained from the subject.

133. The method of claim 132, further comprising comparing the expression level of phosphorylated Merlin in the sample to the expression level of phosphorylated Merlin protein in normal tissue, or cancerous tissue with a known metastatic potential.

134. The method of claim 133, wherein an increase in the level of expression of phosphorylated Merlin is indicative of the presence or metastatic potential of the breast cancer.

135. The method of claim 132, further comprising measuring the expression level of a nucleic acid encoding OPN or the expression level of OPN protein in the sample.

136. The method of claim 135, wherein an increase in the expression level of a nucleic acid encoding OPN or expression level of OPN protein relative to a pre-determined expression level of a nucleic acid encoding OPN or expression level of OPN protein is indicative of the presence or metastatic potential of the breast cancer.

137. The method of claim 132, wherein the breast cancer comprises an infiltrating ductal carcinoma (IDC) or a distant metastasis.

138. The method of claim 132, wherein the phosphorylated Merlin protein is phosphorylated at Threonine 230, Serine 315, or at both residues.

139. The method of claim 132, wherein the subject is mammalian.

140. A method for evaluating the presence, absence or metastatic potential of a breast cancer in a subject comprising measuring the expression level of a nucleic acid encoding OPN or the expression level of OPN protein in a sample obtained from the subject.

141. The method of claim 140, further comprising comparing the expression level of a nucleic acid encoding OPN or the expression level of OPN protein in the sample to the expression level of a nucleic acid encoding OPN or the expression level of OPN protein in normal tissue, or cancerous tissue with a known metastatic potential.

142. The method of claim 141, wherein an increase in the level of expression of a nucleic acid encoding OPN or the level of expression of OPN protein is indicative of the presence or metastatic potential of the breast cancer.

143. The method of claim 140, wherein the breast cancer comprises an infiltrating ductal carcinoma (IDC) or a distant metastasis.

144. A method for identifying a therapeutic compound comprising:
contacting a target cell with a test compound, wherein the cell comprises a breast cancer cell; and
determining whether the test compound significantly changes the level of Merlin protein.

145. The method of claim 144, further comprising determining whether the test compound decreases the expression level of a nucleic acid encoding OPN or OPN protein.

146. A method for identifying a therapeutic compound comprising:
contacting a target cell with a test compound, wherein the cell comprises a breast cancer cell; and
determining whether the test compound significantly changes the expression level of a nucleic acid encoding OPN or the level of expression of OPN protein.

147. A kit for evaluating the presence, absence or metastatic potential of a breast cancer in a subject comprising a detection reagent that binds to Merlin protein.

148. The kit of claim 147, wherein the subject is mammalian.

149. A method for evaluating the presence, absence, or metastatic potential of a cancer in a subject comprising: measuring the expression level of at least one microRNA in a sample obtained from the subject, wherein the microRNA comprises at least about 80% identity to a sequence selected from the group consisting of SEQ ID NO.s:01-74, and a fragment comprising at least 10 consecutive nucleotides thereof.

150. A method for identifying a therapeutic compound comprising:
contacting a target cell with a test compound; and
determining whether the test compound significantly changes the level of at least one microRNA, wherein the microRNA comprises at least about 80% sequence identity to a sequence selected from the group consisting of SEQ ID NO.s:01-74, and a fragment comprising at least 10 consecutive nucleotides thereof.

151. A kit for evaluating the presence, absence or metastatic potential of a cancer in a subject comprising a detection reagent that binds at least one microRNA comprising 80% identity to a sequence selected from the group consisting of SEQ ID NO.s:01-74, a sequence complementary to any one of SEQ ID NO.s:01-74, and a fragment comprising at least 10 consecutive nucleotides thereof.

152. A method of treating breast cancer comprising administering a therapeutically effective amount of an agent which increases the expression level of Merlin protein to a subject having breast cancer.

153. The method of claim 152, wherein the agent is a nucleic acid encoding Merlin or fragment thereof.

154. The method of claim 152, wherein the agent reduces the extent of Merlin phosphorylation.

155. The method of claim 152, wherein the agent reduces phosphorylation of Merlin at residue Threonine 230, at residue Serine 315, or at both residues.

156. The method of claim 152, wherein the agent reduces the extent of Merlin ubiquitination.

157. The method of claim 152, wherein the agent reduces the expression level of a microRNA that targets Merlin.

158. The method of claim 157, wherein the microRNA is selected from the group consisting of SEQ ID NO:01, SEQ ID NO:17, SEQ ID NO:19, SEQ ID NO:20, SEQ ID NO:22, SEQ ID NO:23, SEQ ID NO:24, SEQ ID NO:27, SEQ ID NO:30, SEQ ID NO:34, SEQ ID NO:44, SEQ ID NO:45, SEQ ID NO:50, SEQ ID NO:58, AND SEQ ID NO:59.

159. The method of claim 152, wherein the agent comprises an isolated nucleic acid selected from a small hairpin RNA (shRNA), a small interfering RNA (siRNA), a micro RNA (miRNA), an antisense polynucleotide, and a ribozyme.

160. The method of claim 152, wherein the subject is mammalian.

161. A method of treating breast cancer comprising administering a therapeutically effective amount of an agent which decreases the expression level of a nucleic acid encoding OPN or the expression level of OPN protein to a subject having breast cancer.

162. The method of claim 161, wherein the agent comprises an isolated nucleic acid selected from a small hairpin RNA (shRNA), a small interfering RNA (siRNA), a micro RNA (miRNA), an antisense polynucleotide, and a ribozyme.

163. The method of claim 161, wherein the nucleic acid comprises a sequence encoding OPN or a fragment thereof, a sequence encoding antisense OPN or a fragment thereof, or an antisense nucleic acid complementary to a sequence encoding OPN or a fragment thereof.

164. The method of claim 161, wherein the subject is mammalian.
